(19) United States
(12) Patent Application Publication
(10) Pub. No.: US 2026/0092246 A1
(43) Pub. Date: Apr. 2, 2026

(54) PLATFORM FOR ANTIMICROBIAL SUSCEPTIBILITY TESTING AND BACTERIAL IDENTIFICATION

(71) Applicant: ASTRADX, INC., Chestnut Hill, MA (US)

(72) Inventors: Jonathan Florez, Austin, TX (US); Pragun Goyal, Cambridge, MA (US)

(21) Appl. No.: 19/108,947

(22) PCT Filed: Sep. 5, 2023
(86) PCT No.: PCT/US2023/032008

§ 371 Date: Mar. 5, 2025

Related U.S. Application Data
(60) Provisional application No. 63/374,591, filed on Sep. 5, 2022.

(51) Int. Cl. C12M 1/34; C12M 1/26; C12M 1/32; C12Q 1/04; C12Q 1/18; G06V 10/147; G06V 10/26; G06V 10/764; G06V 20/69

(52) U.S. Cl. CPC C12M 41/36; C12M 23/12; C12M 33/00; C12Q 1/04; C12Q 1/18; G06V 10/147; G06V 10/26; G06V 10/764; G06V 20/693; G06V 20/695; G06V 20/698

(57) ABSTRACT

Devices for performing growth detection, identification and antimicrobial susceptibility testing (AST) functions in connection with a microbial species to achieve sample-to-answer results are disclosed. Methods of performing microbial growth, microbial species identification, and AST using such devices are also disclosed.

### FIELD OF TECHNOLOGY

Aspects relate generally to the rapid detection of bacteremia and fungemia (bloodstream infection) in patients presenting with signs and symptoms to rapidly detect, identify and perform direct antimicrobial susceptibility testing (AST) on organisms associated with infection. Testing is performed directly from a blood sample or other human, animal, or environmental without the need for standard microbial culture. Results are available within 8 hours from the time of blood collection. Rapid organism growth detection and identification can also be performed from a variety of pharmaceutical components and end products, providing a major advantage to the pharmaceutical industry for rapid identification and quantitation of organism bio-burden.

### BACKGROUND

Bloodstream infections are responsible for a fifth of all deaths worldwide, including a third of all hospital deaths in the United States, with an inpatient mortality rate of nearly one in four and are responsible for over 11 million deaths each year. Bloodstream infection is also the single most expensive inpatient condition, costing $42 billion annually in the United States alone. Survival depends on a patient receiving effective antibiotic therapy as soon as possible. Rapid detection of the causative organism along with the early availability of AST results can positively impact patient care and management and reduce complication and possible death.

Survival depends on treating with the appropriate antibiotics as soon as possible, which in turn requires knowing the identity of the bacterium to a clinically actionable level of specificity. In the United States, the primary organization that sets these levels of actionability is called CLSI, so the most urgent diagnostic goal is to determine CLSI-level bacterial ID. Despite a host of newer technologies, the microscope and gram stain still play a critical role in ID. However, what the eye can see on microscopy is limited, necessitating these other technologies. Unfortunately, they are usually complex and expensive, making them less accessible—a critical problem, especially with antimicrobial resistance rising.

### SUMMARY OF THE INVENTION

In accordance with an aspect, there is provided a device configured to perform growth detection, identification, and antimicrobial susceptibility testing (AST) on a microbial species. The device may include a housing configured to receive a sample plate. The device may include a sample port configured to receive a sample suspected to contain the microbial species. The device further may include a fluid distribution system constructed and arranged to introduce the sample to one or more sample wells of the sample plate. The device may include a sample plate imaging system. The device additionally may include a controller configured to collect data from the sample plate imaging system. The controller, once the data has been collected, may detect growth of the microbial species, identify the microbial species, and perform AST on the microbial species.

In some embodiments, the fluid distribution system may include a gantry, a fluid dispensing head operatively coupled to the gantry, and a pump fluidly connected to the fluid dispending head which includes a stepper motor shaft with an absolute-position magnetic encoder. The fluid distribution system may be constructed and arranged to facilitate digital growth detection.

In some embodiments, the sample plate imaging system may include a camera configured with optics, the camera connected to the fluid dispensing head, a light source. and detector array. In certain embodiments, the wherein the detector array may be a photodetector, e.g., a charge coupled device (CCD), a complementary metal oxide semiconductor (CMOS) detector, or a photodiode.

In further embodiments, the device may include a user interface, e.g., a screen, e.g., a touchscreen. In some embodiments, the controller may be configured to transmit information pertaining to growth, identification, and/or AST to a user, e.g., via the user interface.

In further embodiments, the device includes a heater, e.g., disposed beneath the sample plate or between the sample plate and the optics, to facilitate preparation of a sample.

In some embodiments, the device may be further constructed and arranged to enable Gram staining on the sample.

In some embodiments, the device is may be constructed and arranged to perform all three functions in less than about eight hours. In certain embodiments, the device may be constructed and arranged to perform all three functions in less than about six hours. For example, the device can perform AST in less than about one hour. In some embodiments, the device may use a same approach for both growth detection and AST. In some cases, the identification of the microbial species is based on a stainless approach, e.g., without the use of Gram staining.

In some embodiments, the microbial species may include a bacterial species. In some embodiments, the microbial species may be selected from the genus Acinetobacter, Escherichia, Klebsiella, Pseudomonas, Enterococcus, Streptococcus, and Staphylococcus. In some embodiments, the microbial species may fall within one of the following groups of bacteria: Enterococci, Staphylococci, Enterobacterales, Acinetobacter baumannii, and Pseudomonas aeruginosa. In further embodiments, the microbial species may include a nonbacterial species of fungus, mycobacteria, stool parasite, blood parasite or tissue parasite.

In some embodiments, the sample may be a whole blood sample of a subject. In some embodiments, the sample may be a blood component, e.g., plasma, of a subject. In some embodiments, the sample may be a non-blood biological fluid sample of a subject. In further embodiments, the sample may be associated with a pharmaceutical manufacturing component or finished end product.

In some embodiments, the device may be configured to perform one or more of growth detection, identification and AST on a second microbial species.

In accordance with an aspect there is provided a kit for performing growth detection, identification, and antimicrobial susceptibility testing (AST) on a microbial species. The kit may include any device as disclosed herein and a sample plate.

In some embodiments, the sample plate may include a first portion for growth detection and a second portion for AST. In a segregated sample plate, one or more wells in the second portion of the sample plate may be preloaded with a freeze-dried antibiotic. In some embodiments, the antibiotic may be preloaded according to a serial dilution scheme for AST.

In some embodiments, the sample plate may contain a region for performing sample smears. In some embodiments, one or more wells of the sample plate may be defined by a geometry selected to facilitate sensitivity. In some embodiments, the sample plate may contain 384 or 1536 wells.

In further embodiments, the kit may include a source of a growth media or a detection amplifier. In further embodiments, the kit may include a source of a Gram stain. In further embodiments, the kit may include a sample bottle.

In accordance with an aspect, there is provided a method of detecting growth of a microbial species. The method of detecting growth of a microbial species may include providing a sample suspected to contain the microbial species. The method of detecting growth of a microbial species may include acquiring a time series of images of the sample. The method of detecting growth of a microbial species further may include detecting growth of the microbial species via time dependent change pertaining to at least one of light and color across the time series of images.

In some embodiments, detecting growth may include detecting growth using RGB-sensitive imaging sensors in a device, e.g., a device as disclosed herein. Growth of a microbial species may be interpreted as a statistically significant change from a baseline pertaining to at least one of light and color. In some embodiments, a decrease in light and/or a change in color across the time series of images may be interpreted as growth of the microbial species.

In some embodiments, methods of detecting growth of a microbial species may include acquiring a time series of images for each well of a sample plate and detecting growth in each well. In some embodiments, detecting growth further may include comparing one or more of: the time series of images among the wells, between the wells and controls, or between wells a reference time series. In some embodiments, methods of detecting growth of a microbial species may include comparing data across multiple wells to increase sensitivity of growth detection.

In some embodiments, growth detection of microbial species may be achieved in a duration of under five hours. In some embodiments, a number of wells of a sample plate, e.g., at least one well, with observable growth may be used to determine a number of replication-competent microbial cells.

In some embodiments, methods of detecting growth of a microbial species further may include quantifying a bioburden of the sample based on growth detection. In some embodiments, methods of detecting growth of a microbial species further may include one or more remedial actions based on a quantitation of bioburden.

In some embodiments, methods of detecting growth of a microbial species further may include subjecting the microbial species to identification and/or AST upon detecting growth.

In certain embodiments, methods of detecting growth of a microbial species further may include detecting polymicrobial infection by comparing the time series of images between wells of a sample plate. The presence of polymicrobial infection may be evaluated based on a difference in color or doubling time across wells of the sample plate, e.g., well plate, e.g., microwell plate.

In some embodiments, methods of detecting growth of a microbial species further may include processing the time series of images to increase a quality thereof to facilitate earliest possible detection of growth.

In further embodiments, methods of detecting growth of a microbial species may include adding growth media or a detection amplifier to the sample. The sample may be any suitable sample, such as a whole blood sample, blood component sample, other bodily fluid, a product associated with a pharmaceutical manufacturing component or finished end product, a filter membrane, or any other sample where microbial species can be collected and analyzed.

In some embodiments, the method may be characterized by digital growth detection.

In accordance with an aspect, there is provided a method of identifying a microbial species. The method of identifying a microbial species may include imaging a sample that has a plurality of microorganisms of the microbial species to obtain a series of polymicrobial images. The method of identifying a microbial species may include segmenting the polymicrobial images. The methods of identifying a microbial species further may include measuring parameters of each segmented microorganism to provide a multidimensional distribution of measured parameters. The method of identifying a microbial species additionally may include classifying the microbial species based on the multidimensional distribution of measured parameters.

In some embodiments, the measured parameters may pertain to one or more of size, shape, intrinsic color, arrangement and other morphological properties of the microbial species. For example, at least one of the one or more measured parameters may be selected from the group consisting of: width, length, interior density, membrane thickness, color heterogeneity, color concentration, curvature, tapering, aspect ratio, and concavity. In specific embodiments, the method of identifying a microbial species may include inherent color data associated with the microbial species without staining, i.e., without Gram staining.

In some embodiments, classification of the microbial species may involve use of machine learning, e.g., a machine learning algorithm, trained with at least one of the following modalities: (i) unstained slides imaged with direct light; (ii) unstained slides imaged with indirect light, e.g., darkfield microscopy; and (iii) pairing pre- and post-stained images. In certain embodiments, classifying the microbial species may involve a probability distribution. In certain embodiments, classifying may be performed via a hierarchical approach. In certain embodiments, classification may be performed via majority rule, decision tree, or relative entropy between an observed tally and a reference distribution of a known microbial species.

In some embodiments, the method of identifying a microbial species may include distinguishing gram positive bacteria from gram negative bacteria with a first confidence score. Using this first distinction, the species of each bacteria may be then identified with a second confidence score.

In further embodiments, methods of identifying a microbial species may include filtering the images, e.g., using a colored filter. For example, a blue filter can be applied to all collected images to provide for image sharpening. In some embodiments, methods of identifying a microbial species may include collecting images of each sample at multiple focal lengths of the optical system. As a non-limiting example, images of each sample are taken at a series of small or fine-grained focal-distance intervals. In some embodiments, the fine-grained focal-distance intervals may be from about 0.1 μm to 2 μm. In specific embodiments, the focal-distance intervals may be 0.5 μm. In some embodiments, methods of identifying a microbial species may include acquiring a plurality of images at each focal length and combining the images.

In further embodiments, methods of identifying a microbial species may include selecting an image having a greatest value of one or more quality metrics for measurement. The quality of the selected image can be improved by removing or smoothing numerical noise prior to segmentation. In some embodiments, methods of identifying a microbial species may include

performing dimensionality reduction, e.g., PCA or UMAP, on the multidimensional distribution of measured parameters.

In some embodiments, measuring 100 randomly selected segmented microorganisms may be sufficient to identify the microbial species with a confidence of about 93-97%.

In some embodiments, a sample of the microbial species may originate from a growth detection study. The growth detection study may have been performed using the device as disclosed herein or a same or different sample plate.

In further embodiments, methods of identifying a microbial species may include identifying a second microbial species.

In accordance with an aspect, there is provided a method of performing growth detection, identification and antimicrobial susceptibility testing (AST) on a microbial species. The disclosed method may be configured to perform all three functions, i.e., growth detection, identification and antimicrobial susceptibility testing, in less than about eight hours.

In some embodiments, one or both of the growth detection of the microbial species and identification of the microbial species may be performed using the methods disclosed herein.

In some embodiments, AST may be based on a differential growth detection method, e.g., as disclosed herein. For example, the AST may involve dilution temporal modeling (DTM). Using methods disclosed herein, AST may achieve categorical agreement to a reference method in under one hour, e.g., under 1 hour. In some embodiments, the reference method for AST comparison may include broth microdilution.

In some embodiments, the AST may be performed with respect to an antibiotic selected from cefepime, meropenem, ciprofloxacin, and gentamicin.

In some embodiments, results pertaining to AST may be reported to an operator, a laboratory information system, and/or an electronic medical record, e.g., transmitted by the device as disclosed herein or viewed using a user interface on the device as disclosed herein.

In some embodiments, AST may be performed once a microorganism has been identified at a categorical level but before the microorganism has been identified at the species level. In some embodiments, each of growth detection, identification, and AST may be performed in a single device, e.g., a device as disclosed herein.

In accordance with an aspect, there is provided a quality control method for a pharmaceutical manufacturing process. The quality control method may include performing the method of detecting growth of a microbial species, e.g., using any device or method disclosed herein, on a sample containing a pharmaceutical component or finished end product to assess a bioburden thereof.

In some embodiments, the quality control method for a pharmaceutical manufacturing process may include accepting or rejecting the pharmaceutical component or finished end product based on comparison of the assessed bioburden to a threshold value.

In accordance an aspect, there is provided single device configured to perform growth detection, identification and antimicrobial susceptibility testing (AST) functions on a microbial species.

In some embodiments, the device is configured to perform all three functions in less than about eight hours.

In some embodiments, the device is configured to perform all three functions in less than about six hours.

In some embodiments, the device performs AST in less than about one hour.

In some embodiments, the device uses a same approach for both growth detection and AST.

In some embodiments, the identification function is based on a stainless approach via machine learning.

In some embodiments, the microbial species comprises at least one species from the genus Acinetobacter, Escherichia, Klebsiella, Pseudomonas, Enterococcus, Streptococcus, and Staphylococcus.

In accordance with an aspect, there is provided a method of identifying a microbial species via machine learning, wherein the method involves a stainless approach.

In accordance with an aspect, there is provided a method of detecting growth of a microbial species. The method may include acquiring a plurality of images of a microbial sample using an image collection system. The method may include sending or transmitting one or more of the plurality of images to an image analysis system comprising a non-transitory computer-readable medium storing thereon sequences of computer-executable instructions for determining growth of the microbial species from one or more of the plurality of images of the microbial sample by manipulating data corresponding to a pixel intensity of one or more regions of one or more of the plurality of images to a hybrid model representative of a growth dynamic of the microbial species.

In accordance with an aspect, the is provided a method of performing growth detection, identification and antimicrobial susceptibility testing (AST) on a microbial species, wherein the method is configured to perform all three functions in less than about eight hours.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates direct from blood (as representative direct starting material)-to-answer time in accordance with one or more embodiments.

FIGS. 2A-2C illustrate schematics of a device for AST in accordance with one or more embodiments. FIG. 2A illustrates a front view of the device. FIG. 2B illustrates a side cutaway view of the device. FIG. 2C illustrates front cutaway view of the device.

FIG. 3 illustrates a workflow for AST in accordance with one or more embodiments.

FIGS. 4A-4B illustrate results from experiments demonstrating growth in under five hours (FIG. 4A) and under four hours (FIG. 4B) in accordance with one or more embodiments.

FIGS. 5A-5C illustrate results from the growth of five different bacterial species and reduced dimensionality visualization to aid in the identification of unknown samples. FIG. 5A illustrates the segmented cells for each of the five different bacterial species. FIG. 5B illustrates the reduced dimensionality relationship of the five different bacterial species using UMAP. FIG. 5C illustrates the confidence in identifying unknown samples.

FIGS. 6A-6C illustrate comparisons between AST performed using a reference method and using methods disclosed herein. FIG. 6A is a 3D plot for E. coli growth as a function of meropenem concentration. FIG. 6B is a 3D plot for E. coli growth as a function of cefepime concentration. FIG. 6C shows the equations used to model growth.

FIG. 7 illustrates the AST of a number of organisms in standard antibiotics using methods disclosed herein.

FIGS. 8A-8C illustrate use of a fluid handling system in accordance with one or more embodiments. FIG. 8A illustrates dispensing of fluid into standard well plates. FIG. 8B illustrates the filling of 10 μL of liquid into the well plates. FIG. 8C illustrates the standard deviation and standard error for the fluid handling system across different dispensing volumes.

FIG. 9 illustrates bacterial identification techniques in accordance with one or more embodiments.

FIGS. 10A-10E illustrate a proposed AST workflow in accordance with one or more embodiments. FIG. 10A illustrates image acquisition. FIG. 10B illustrates a first step of image processing. FIG. 10C illustrates segmentation of the individual cells in an image. FIG. 10D illustrates fingerprint analysis of segmented cells. FIG. 10E illustrates statistical comparisons to reference images for identification.

### DETAILED DESCRIPTION

In accordance with one or more embodiments, devices and methods for both antibiotic susceptibility testing and microbial identification are disclosed. In some aspects, both objectives may be accomplished with a single device that is relatively inexpensive and compact. Beneficially, the disclosed systems and methods may achieve both objectives in a relatively short period of time, estimated to be 8 hour or less. Workflow may be significantly streamlined and errors reduced. Whole blood or other biospecimens may be directly introduced to the disclosed devices, and within 4-8 hours pathogenic organisms are identified and AST results are generated. Ineffective-treatment-related mortality may be halved.

Different antibiotics are used for treating different bacteria. For example, vancomycin is used empirically to cover S. aureus but not Escherichia coli or other Enterobacterales, which are generally resistant. Conversely, broad-spectrum β-lactam antibiotics empirically cover Enterobacterales but not S. aureus. P. aeruginosa and Acinetobacter baumannii need still other antibiotics. Bacterial identification makes it possible to narrow therapies, tailoring it to the offending bacterial species or group. Targeted therapy lowers the risk of life-threatening side effects such as superinfection with Clostridioides difficile, which results from disturbance of the body's normal flora by broad-spectrum antibiotics.

Antimicrobial susceptibility testing (AST) is the process of determining which antibiotics a bacterium will respond to. Typically to perform AST, bacteria are grown at different antibiotic concentrations to determine the lowest concentration that inhibits growth; this is known as the minimum inhibitory concentration or MIC. If the MIC is below a certain breakpoint (set by the CLSI or similar standards-setting organization), the organism is considered susceptible, meaning the antibiotic can be used. The gold standard AST method is phenotypic AST, in which the organism is tested for how well it grows in the presence of an antibiotic. Phenotypic AST is preferred over genotypic AST, an indirect method in which bacterial genetic material is tested for the presence of DNA sequences that correlate with phenotypic behavior. Phenotypic AST is the mainstay of care.

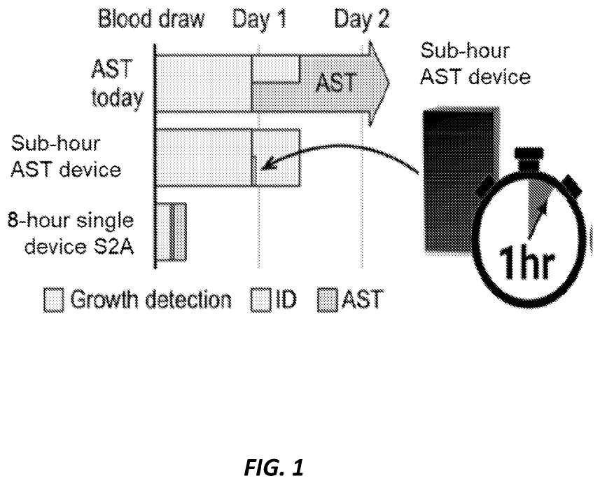

An important factor for patient survival is the time it takes from blood draw to AST results: the sample-to-answer-time (S2A). Mortality rises 7.6% every hour a patient is on the wrong antibiotic. Yet today, the median S2A is two days as illustrated in FIG. 1, with a long tail that can stretch into weeks for multidrug-resistant organisms, which are on the rise. Given that the average inpatient stay for a bloodstream infection is 7.1 days, a long S2A can make AST moot and can mean death for the patient.

Mortality doubles with every day a patient is on ineffective therapy, so a reduction of such magnitude would have great significance. International guidelines require that patients receive a dose of broad-spectrum empiric antibiotics immediately after cultures are drawn. The next dose of antibiotic is typically administered 6-8 hours later. In accordance with one or more embodiments, AST results may be available before the next dose is administered, i.e., within 6-8 hours of cultures being drawn. In accordance with at least some embodiments, S2A does not exceed a standard 8-hour work shift, thus avoiding delays and errors associated with shift changes. A meaningful target S2A may be ≤6-8 hours. Devices and methods described herein meet this target.

The embodiments can detect bacteria in components of pharmaceuticals throughout the manufacturing process. Current methods may take up to 48-72 hours to detect potential bacterial contamination in pharmaceutical components and finished product. A meaningful target for detection of bacteria (bio-burden) can result in more efficient manufacturing saving companies time and money.

In accordance with one or more embodiments, computer vision, signal processing and other technologies may be adapted to the service of same-shift S2A. Computer vision is the application of artificial intelligence/machine learning to image analysis and other optical signals to achieve superhuman speed and/or accuracy. Signal processing as used herein involves the cleanup of time series to detect the earliest possible signs of growth.

In accordance with one or more embodiments, sub-hour AST starting from positive blood cultures may be achieved. In some embodiments, differential growth detection for AST may be implemented involving detection of subtle differences in the darkening of wells as bacteria grow, depending on the antibiotic and its concentration.

In accordance with one or more embodiments, AST may be in line with techniques disclosed in co-pending International (PCT) Application Serial No. PCT/US2022/042509 which is hereby incorporated by reference herein in its entirety for all purposes. In accordance with one or more embodiments, more sensitive sensors and algorithms may be implemented.

Conventionally, it takes a median of 22 hours for blood cultures to turn positive after which organisms must be sub-cultured for organism identification and AST, requiring an addition 12-24 hours. In accordance with one or more embodiments described herein, positive blood cultures may be detected sooner to reduce S2A.

In accordance with one or more embodiments, digital growth detection is disclosed. “Digital” means that the number of viable organisms in the sample can be counted because, in a multiwell format, each positive well begins with no more than a single cell of the pathogen. For example, the microtiter plate format enables digital growth detection, yielding micro-cultures that are pure because each such micro-culture starts from a single bacterial cell. In at least some embodiments, sensitive ab initio growth detection may be achieved. In some non-limiting embodiments, an approach for AST (such as those disclosed in the co-pending application incorporated above) may similarly be used for growth detection as discussed herein in many human and solution matrices perform pathogen or other organism identification (ID), such as bacterial ID. In the gold-standard version of phenotypic AST, bacteria are grown in serial dilutions of antibiotic—1 μg/mL, 2 μg/mL, 4 μg/mL, etc.—to find the lowest concentration at which bacteria fail to grow. This lowest concentration is the antibiotic's minimum inhibitory concentration (MIC). In general, a low MIC in the laboratory means the drug will be effective in the patient; however, the cutoff or breakpoint for susceptibility vs. resistance differs according to ID. In some cases bacteria may share the same breakpoint. Current laboratory standards require bacterial identification prior to reporting an AST result, making organism identification critical to the clinical process of identifying causative agents of infection. Since ID is critical to AST reporting it is essential that bacterial identification be included in the S2A device.

Although bacterial ID can be done rapidly, taking only several minutes to a few hours with the right combination of mass spectrometry, PCR, and/or biochemical techniques, these approaches require not only the purchase and maintenance of hundreds of thousands of dollars' worth of equipment but also in most cases growth of a pure culture which can take 12-24 hours.

In accordance with one or more embodiments, computer vision may be used for ID. Related architectures and pipelines may be tailored in accordance with one or more embodiments rather than simply borrowing from existing (i.e., non-medical) neural networks.

In accordance with one or more embodiments, color data of unstained organisms (i.e., without gram stain or other stains) from a sensor may be used for identification. For example, Pseudomonas aeruginosa and E. coli are both Gram-negative rods but Pseudomonas aeruginosa famously has a green-to-purple appearance, while E. coli is usually off-white-to-yellow. Just as colony color and bacterial cell morphology have long helped microbiologists to presumptively ID bacteria, they can help computer vision ID more specifically,: and faster (as fast as the device can prepare a slide; a matter of minutes) without the need for gram stain or any other stain. On-device bacterial ID is an enabling innovation for rapid S2A.

Gram stains have been a 140-year-old mainstay of microbiology allowing microscopic examination to differentiate bacteria. The stains used in the gram staining procedure—there are two: one that makes gram-positive organisms appear purple and one that makes gram-negatives appear pink—help make the morphology of individual bacterial cells (e.g., their size and shape), and their arrangement relative to each other, visible to human eyes with the aid of a microscope. Definitionally, they also help distinguish between gram-positive and gram-negative organisms. The staining difference reflects a useful biological difference between organisms, which affects potential antibiotic treatment options. However, there are two important trade-offs. First, any information related to an organism's inherent (i.e., un-/pre-stained) color is lost when they are gram stained. It is well documented that organism colonies can have distinctive colors, which can be informative for identification.

The described embodiments may use color as part of a machine learning approach to identify organisms. Second, importantly, even though gram staining is a straightforward procedure, it involves performing several staining and washing steps using several chemicals and requires interpretation by trained clinical microbiologists. Inter-operator is a well described problem. Therefore, there would be advantages to being able to perform ID without needing to stain.

Unstained cells have characteristic colors. Gram staining, in practice, can mask some of the valuable information it provides. From a macroscopic perspective, the color of bacterial colonies has been a key indicator of ID since gram staining was first described. Microscopically, the color of unstained bacteria can be seen by computer. Dispensing with Gram staining therefore upgrades Gram staining's binary purple-pink color for a richer intrinsic palette that should be even more informative for bacterial identification. The gram stain itself provides largely redundant information: most clinically important gram-negative bacteria are rod-shaped and most gram-positives are cocci; exceptions differ in arrangement (e.g., Neisseria) and/or shape and size (e.g., Clostridia). Without wishing to be bound by any particular theory, it is

believed that the same bacterial cell-wall physiology that gives rise to the purple vs. pink color upon Gram staining will be detectable from optical measurement. For example, low-magnification electron microscopy (EM) has been shown to predict Gram stain. Machine learning has further been used to predict complex hematoxylin and eosin (H&E) staining patterns from unstained human tissue, suggesting that optical measurement can likewise predict gram-stain patterns from unstained images.

In accordance with one or more embodiments, photomicrographs of unstained slides may be used for identification. Deep networks are expert at noticing subtle differences in light and color that will allow them to identify bacteria from unstained slides. In some embodiments, three (related) modalities may be used to train with: (i) unstained slides imaged with direct light; (ii) unstained slides imaged with indirect light, a method known as darkfield microscopy; and (iii) pairing pre- and post-stained images, a method that has been used in pathology to predict staining patterns but not to machine-learn what the staining shows, and not for bacterial, fungal, or other microbiological identification.

In accordance with one or more embodiments, identification may be achieved from unstained slides, especially making use of color-both the faint color of individual bacterial or other cells on the slides and color as determined by non-slide-based methods, e.g., the color of organisms growing in broth (liquid media).

Individual bacteria on gram stain (or other stains) are described by size, shape, and color. A typical broth-culture slide contains many thousands of organisms. This plentitude offers the opportunity for statistical learning based on bacterial size, shape, and other morphological parameters. Beginning with an image, the overall approach is to identify and isolate each the image of each bacterial (or other) cell using image segmentation. (Non-cell debris from blood, urine, or other matrix can also be segmented.)

In accordance with one or more embodiments, each bacterium may be measured. Regarding bacterial measurement in general, existing measurements that exist as part of descriptions in the research literature are broad ranges (e.g. 0.5-1 μm×0.8-1.5 μm-two-fold ranges in all dimensions), disagree from source to source, are unreferenced, and reflect received wisdom that generally appears to trace back to textbooks from a century before high-throughput measurements were available.

Regarding the specific measurements envisioned as part of the current disclosure, Bacteria are generally convex shapes, classically (and most often) somewhere between a rod (bacillus) and a sphere (coccus). They differ in particulars like the curvature of their ends (boxcar), whether and/or to what extent they taper (coryneform), and their aspect ratios (coccobacillary, fusiform, filamentous). Equations exist for parametrically, i.e., smoothly, interpolating among all these shapes. Cassini ovals and Cartesian ovals are common examples. Harmonics are another equation type used for shape interpolation. The equation:

$$1 = \frac{{x}^{2}}{{a}^{n}} + ( \frac{{y}^{2}}{{b}^{m}} ) * f ( x )$$

with different forms of f(x) (e.g., ƒ(x)=1+kx/1−kx or ƒ(x)=e^{kx} for different k, also has this property. There are also equations for expressing bacterial curvature (e.g., Campylobacter) and concavity (e.g., Bifidobacterium).

In accordance with one or more embodiments, each bacterium may be fitted using one or more of these equations and recording the parameters. Simple width and length may be measured, as well as parameters that reflect the complexity and differential density of the interior of the cell, the thickness of the membrane (in darkfield microscopy), and concentrations and/or heterogeneity of the color of each of these.

In accordance with one or more embodiments, a distribution or histogram of these parameter may be created for each slide. Each slide is thereby converted into a multidimensional distribution of parameters, one dimension for each parameter measured. Instead of “E. coli are 0.5-1 μm×0.8-1.5 μm,” there is a multidimensional distribution that describes precisely how the lengths and widths, as well as the other parameters mentioned above, are distributed for the hundreds or thousands of individual cells on the slide.

In accordance with one or more embodiments, robust representations of each species and strain may be learned. At its simplest, learning consists of a database that maps each species (or strain) to its multidimensional distribution. Multidimensional distributions can be considered as point clouds, where each point is a single cell and the location of the point is given by its measurements. Each species would have a cloud of a different shape with different regions of density. Given an unknown organism, Steps 1-3 disclosed herein can be performed, create its cloud, find the most similar cloud in the database, and assign the corresponding bacteria as the identity.

In another implementation, one would learn a representation or model of the cloud, using for example deep learning, maximum-entropy modeling, or some other approach, and use the resulting model to assign identity to an unknown. In such an implementation, the key is that the model can output a probability that a given bacterial cell belongs to the cloud, and the set of probabilities for all cells can be used for an aggregate or “grand” probability to assign an identity or determine whether more than one identity is present, in some embodiments. Thus, given a slide of an unknown organism, for example, one would segment and measure each cell, and then for each cell, ask a of model of, for example, E. coli, what the probability is that that unknown cell would be found in the E. coli cloud. The result would be a set of probabilities—i.e., a probability distribution—for all the cells present, for the unknown being E. coli. One would generate such distributions for each organism in the database—E. coli, P. aeruginosa, S. aureus, etc.—and whichever organism's model gave the highest probabilities, that organism would be assigned as the unknown's identity.

In accordance with one or more embodiments, an approach to microbial identification via machine learning is disclosed that requires no gram staining or other staining.

In accordance with one or more embodiments, identities may be machine learned from combination of (i) unstained light-microscopy images and/or (ii) darkfield images and/or (iii) color in growth experiments. Darkfield microscopy images may also still have color information which could be used.

In accordance with one or more embodiments, non-bacterial organisms could be identified. These may include fungi like yeast and Aspergillus, mycobacteria like Mycobacterium tuberculosis, stool parasites like pinworm, blood parasites like malaria and Babesia, and tissue parasites.

In accordance with one or more embodiments, alternatives to deep learning could be implemented, for example, parameterized learning in which a defined set of parameters may be varied.

In accordance with one or more embodiments, individual organisms in a polymicrobial image may be segmented and classified.

In accordance with one or more embodiments, color from microtiter growth experiments may be measured. Turbidity can be thought of as a monochrome measurement. So can optical density at a specific wavelength; for example, 600 nm (yellow) is the most commonly used wavelength, which is where E. coli has its peak absorbance. The embodiments disclosed herein use color, measured for example by combining signals from specific sensors (e.g., R, G, B). Use of color in the disclosed embodiments also includes color changes over time, which may be represented as a color wheel, in which hue is represented circumferentially, e.g., by the angle around the wheel, and time is represented by the radius, with the center marking the start (i.e., t=0). As the organism grows over time, the change in color of the broth is represented by a line, not necessarily a straight line, from the center of the color wheel toward the edge. The final color may relate to where the line ends up when it hits the edge of the color wheel at the end of whatever period of time growth is being observed for. A trend in color may also be observed wherein the broth might start out as its default color before becoming green, coincident with the beginning of pigment production, the idea being the time-dependent pattern is useful. Statistics and computer/mathematics-aided amplification of human-imperceptible colors/color changes are used to assign a color at each point in time.

In accordance with one or more embodiments, the computer vision approach that enabled sub-hour AST direct from blood without the need for culture is enhanced to achieve both fast growth detection and perform identification. The platform technology of computer vision enables all three steps in a single elegant solution, as it simplifies engineering and thereby enables an affordable final product in the fight against bloodstream infections. The ability to detect and identify bacteria rapidly provides an important solution for the pharmaceutical industry as an improved method to determine bio-burden.

In accordance with one or more embodiments, the analysis of unstained images, e.g., collected using microscopy, of bacterial species includes three steps: a cleanup step, a segmentation step, and a fingerprinting step. During the image cleanup, one or more machine learning algorithms are used to increase the quality of the collected images, such as by removing or smoothing numerical noise in the collected pixels. In the segmentation step, individual bacterial cells in an image are identified using image processing that upsamples each pixel to decrease the contributions of debris in each image, effectively segmenting each identified bacterial cell away from the remaining image. “Segmentation” is a term of art in image processing that refers to isolating the pixels that correspond to an object from all other pixels in the image (i.e., from the background and all other objects). And in the fingerprinting step,

quantitative measurements of cell size, shape, intrinsic color, and arrangement are performed on each segmented bacterial cell to identify the bacterial species.

### Devices

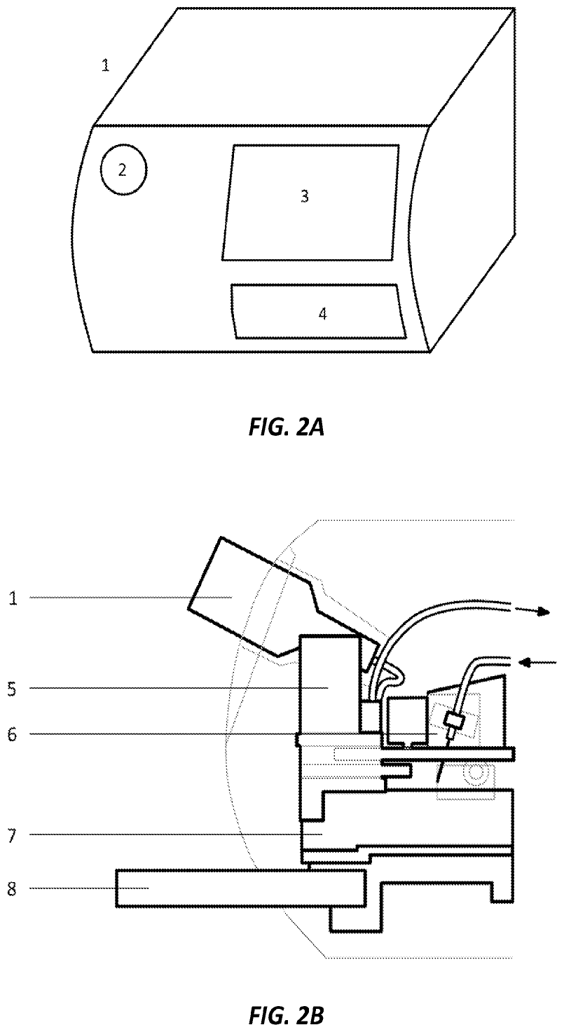

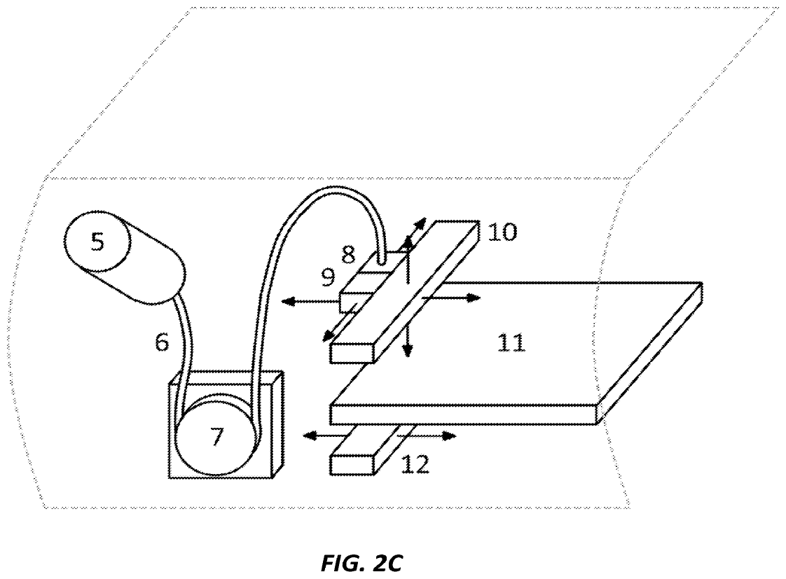

In accordance with an aspect, there is provided a device for the detection of bacterial growth and antibacterial susceptibility testing (AST). An embodiment of a device is illustrated in FIGS. 2A-2C. With reference to FIGS. 2A-2C, the device includes sample input 1, user interface 2, and housing 3. Integrated into the housing 3 is a door 4 that opens to permit a user or operator to insert a standard well plate into the device. The sample input 1 is designed to accept standard phlebotomy sample bottles but other types of sample inputs, e.g., injections, are within the scope of this disclosure. As illustrated, the user interface 2 is a touchscreen, but can also be any type of suitable display output, such as a non-capacitive LCD/LED screen with dedicated controls, e.g., a keypad, keyboard, or a mouse, a cathode ray tube (CRT) screen, or any other suitable display. Alternatively, the user interface can be an external display that connects to the device using standard display connections, e.g., universal serial bus (USB), DVI, HDMI, VGA, DisplayPort, or any other suitable display standard. When the user interface is external, the device may include connections for external controls, such as a keyboard and/or a mouse.

With continued reference to FIGS. 2A-2C, the device includes a gantry 5, a fluid dispensing head 6, and a pump 7. In some embodiments, the device may also include a fluid reservoir for reagents and/or a waste fluid reservoir. The gantry 5 generally includes a frame adapted to act a track that the fluid dispensing head 6 moves in the Cartesian plane. The gantry 5 also includes a motor that is used to actuate the fluid dispensing head 6 along its axes. The fluid dispensing head includes a fluid tube fluidically connected to the pump and a flexible dispensing needle. The tubing and flexible dispensing needle can be any tubing and needle suitable for use in the study of bacterial species, and types of each are known in the art. In general, the tubing is both non-stick and non-toxic to reduce clogging in the fluid dispensing head 6 and to ensure survivability of the bacterial species during various fluid transfers. Operatively coupled to the fluid dispensing head 6 is a camera configured with suitable optics to enable imaging of the bacterial species in each well of a sample plate. In operation, the gantry 5 moves the combined fluid dispensing head 6 and its camera to dispense fluid into each well of a sample plate and image each well. The pump 7 directs fluid from sample input 1 through tubing 9 into the fluid dispensing head 6 for deposition into each well. The pump 7 can also aspirate to remove fluid from each well so it can be collected for further analysis. The pump 7 can be any suitable pump for moving biological fluids. As illustrated in FIGS. 2A-2C, pump 7 is a peristaltic pump but this is only an embodiment and any suitable pump can be used. In further embodiments, the device includes a heater to maintain the samples in the device at a temperature conducive for analysis. The heater may also be used in sample preparation, such as by drying solvents or fixing samples to microscope slides. The heater may be located beneath the sample plate or between the sample plate and the optics but may be located in any suitable location in the device.

With continued reference to FIGS. 2A-2C, when door 4 is opened, a well plate 8 can be inserted into plate holder 12 that sits within a space in the lower portion of the housing 3. When inserted, the well plate 8 is positioned beneath a light source 11 and the combined fluid dispensing head 6 and its camera. Beneath the well plate 8 and the plate holder 12 is a detector array 13 that captures light that passes through the sample plate 8 when illuminated by light source 11. Detector array 13 can be any suitable photodetector, such as a charge coupled device (CCD), a complementary metal oxide semiconductor (CMOS) detector, and a photodiode.

The device includes a controller 10 for performing one or more operations within the device. The controller 10 includes a processor for executing instructions, non-transitory computer readable media for storing instructions to be executed by the processor 10 and for storing collected images and processed results, and a communications module to provide connectivity to the Internet for data transfers and communications within the setting the device is located, e.g., a hospital, clinic, research institution, or industrial workplace. The controller 10 may be implemented using one or more computer systems. The computer system may be, for example, a general-purpose computer such as those based on an Intel CORE®-type processor, an Intel XEON®-type processor, an Intel CELERON®-type processor, an AMD FX-type processor, an AMD RYZEN®-type processor, an AMD EPYC®-type processor, and AMD R-series or G-series processor, or any other type of processor or combinations thereof. Alternatively, the computer system may include programmable logic controllers (PLCs), specially programmed, special-purpose hardware, for example, an application-specific integrated circuit (ASIC) or controllers intended for analytical systems. In some embodiments, the controller C may be operably connected to or connectable to a user interface constructed and arranged to permit a user or operator to view relevant operational parameters of a system as disclosed herein, adjust said operational parameters, and/or stop operation of a system as needed. The user interface may include a graphical user interface (GUI) that includes a display configured to be interacted with by a user or service provider and output status information of the system.

The controller 10 can include one or more processors typically connected to one or more memory devices, which can comprise, for example, any one or more of a disk drive memory, a flash memory device, a RAM memory device, or other device for storing data. The one or more memory devices can be used for storing programs and data during operation of the odor control system and/or the control subsystem. For example, the memory device may be used for storing historical data, operating data. Software, including programming code that implements embodiments of the invention, i.e., deep learning algorithms used for the various steps of image processing disclosed herein and statistical fitting, can be stored on a computer readable and/or writeable nonvolatile recording medium, and then typically copied into the one or more memory devices wherein it can then be executed by the one or more processors. Such programming code may be written in any of a plurality of programming languages, for example, ladder logic, Python, Java, Visual Basic, C, C#, or C++, Fortran, Pascal, Eiffel, Basic, COBOL, or any of a variety of combinations thereof.

The communications module of the controller 10 can include wired communications connections via industry standard connections such as broadband internet connection, e.g., a Local Area Network (LAN) or a Wide Area Network (WAN) using USB, RS-232, RJ-11, RJ-45, i.e., Ethernet, or another wired standard. Alternatively, or in addition, the communications module can include wireless connectivity over a wireless transmission standard, e.g., Wi-Fi, BLUETOOTH®, 5G NR FR2, LTE Cat 1, LTE Cat M1 or Cat NB1 standard. In some embodiments, the controller 10 has as communications module that includes both wired and wireless communication features.

### Kits

The present disclosure further provides a kit including the device as described herein and one or more sample plates, e.g., well plates, e.g., 384 or 1546 well plates. The sample plates provided with the kit can be segmented to provide for a first portion of the plate to be used for growth detection and a second portion to be used to AST. For example, for the wells in the second portion of the plate, one or more of these wells may come pre-filled or preloaded with a mass or volume of an antibiotic, e.g., a freeze dried pellet of an antibiotic. When the wells in the second portion of a sample plate include an antibiotic for AST, the arrangement of the antibiotic may be in a manner for a serial dilution scheme, i.e., increasing or decreasing the concentration of the antibiotic over a regular volume interval. In some embodiments, a sample plate included as part of a kit disclosed herein can include a region of the plate, i.e., a portion of the well, for performing sample smears as disclosed herein.

Kits as disclosed herein can include one or more reagents useful for bacterial species growth detection, identification, or AST. In some embodiments, kits as disclosed herein can include a source of a growth media or a detection amplifier. In some embodiments, kits as disclosed herein can include a Gram staining dye. These reagents can be come in any suitable packaging with the kit. Kits as disclosed herein can include one or more additional components, such as sample bottle, e.g., a vacutainer or other reagent specific blood collection tube.

### Methods

In accordance with an aspect, methods of detecting growth of a microbial species are disclosed. Methods of detecting growth of a microbial species include providing a sample suspected to contain the microbial species. The methods of detecting growth of a microbial species includes acquiring a time series of images of the sample. The methods of detecting growth of a microbial species further include detecting growth of the microbial species via time dependent change pertaining to at least one of light and color across the time series of images.

In some embodiments, detecting growth comprises detecting growth using RGB-sensitive imaging sensors in a device. Growth of a microbial species is interpreted as a statistically significant change from a baseline pertaining to at least one of light and color. For example, a decrease in light and/or a change in color across the time series of images is interpreted as growth of the microbial species.

In some embodiments, methods of detecting growth of a microbial species include acquiring a time series of images for each well of a sample plate and detecting growth in each well. In some embodiments, detecting growth further comprises comparing one or more of: the time series of images among the wells, between the wells and controls, or between wells a reference time series. In some embodiments, methods of detecting growth of a microbial species include comparing data across multiple wells to increase sensitivity of growth detection.

Using methods disclosed herein, growth detection of microbial species is achieved in a duration of under five hours, e.g., under five hours, under four hours, under three hours, under two hours, or under one hour. In some embodiments, a number of wells of a sample plate with observable growth is used to determine a number of replication-competent microbial cells. Without wishing to be bound by any particular theory, the number of wells divided by total volume of sample that went on the plate generally equals the number of replication-competent cells, i.e., colony-forming units (CFUs) per unit volume, with volume in mL.

In some embodiments, methods of detecting growth of a microbial species further include quantifying a bioburden of the sample based on growth detection. As used herein, “bioburden” refers to the quantity and types of native bacterial and fungal flora present on or in a device, substrate, or chemical. Bioburden plays a large role in determining what is necessary to achieve sterility in a given environment. In some embodiments, methods of detecting growth of a microbial species further include one or more remedial actions based on a quantitation of bioburden.

In some embodiments, methods of detecting growth of a microbial species further include subjecting the microbial species to identification and/or AST upon detecting growth.

In certain embodiments, methods of detecting growth of a microbial species further include detecting polymicrobial infection by comparing the time series of images between wells of a sample plate. The presence of polymicrobial infection is evaluated based on a difference in color or doubling time across wells of the sample plate, e.g., well plate, e.g., microwell plate.

In some embodiments, methods of detecting growth of a microbial species further include processing the time series of images to increase a quality thereof to facilitate earliest possible detection of growth. As disclosed herein, growth detection of microbial species is achieved in a duration of under five hours, e.g., under five hours, under four hours, under three hours, under two hours, or under one hour.

In further embodiments, methods of detecting growth of a microbial species include adding growth media or a detection amplifier to the sample. The sample may be any suitable sample, such as a whole blood sample, blood component sample, other bodily fluid, a product associated with a pharmaceutical manufacturing component or finished end product, a filter membrane, or any other sample where microbial species can be collected and analyzed.

In some embodiments, the method is characterized by digital growth detection.

In accordance with an aspect, methods of identifying a microbial species are disclosed. Methods of identifying a microbial species include imaging a sample comprising a plurality of microorganisms of the microbial species to obtain a series of polymicrobial images. Methods of identifying a microbial species include segmenting the polymicrobial images. Methods of identifying a microbial species further include measuring parameters of each segmented microorganism to provide a multidimensional distribution of measured parameters. Methods of identifying a microbial species additionally include classifying the microbial species based on the multidimensional distribution of measured parameters.

In some embodiments, the measured parameters pertain to one or more of size, shape, intrinsic color, arrangement and other morphological properties of the microbial species. For example, at least one of the one or more measured parameters is selected from the group consisting of: width, length, interior density, membrane thickness, color heterogeneity, color concentration, curvature, tapering, aspect ratio, and concavity. In specific embodiments, methods of identifying a microbial species include inherent color data associated with the microbial species without staining, i.e., without Gram staining.

In some embodiments, classification of the microbial species involves use of machine learning, e.g., a machine learning algorithm, trained with at least one of the following modalities: (i) unstained slides imaged with direct light; (ii) unstained slides imaged with indirect light, e.g., darkfield microscopy; and (iii) pairing pre- and post-stained images. In certain embodiments, classifying the microbial species involves a probability distribution. In certain embodiments, classifying is performed via a hierarchical approach. In certain embodiments, classification is performed via majority rule, decision tree or relative entropy between an observed tally and a reference distribution of a known microbial species.

In some embodiments, methods of identifying a microbial species include distinguishing gram positive bacteria from gram negative bacteria with a first confidence score. Using this first distinction, the species of each bacteria is then identified with a second confidence score.

In further embodiments, methods of identifying a microbial species include filtering the images, e.g., using a colored filter. For example, a blue filter can be applied to all collected images to provide for image sharpening. In some embodiments, methods of identifying a microbial species include collecting images of each sample at multiple focal lengths of the optical system. As a non-limiting example, images of each sample are taken at a series of small or fine-grained focal-distance intervals. In some embodiments, the fine-grained focal-distance intervals may be from about 0.1 μm to 2 μm, e.g., about 0.1 μm to 2 μm, about 0.2 μm to 1.8 μm, about 0.3 μm to 1.6 μm, about 0.4 μm to 1.4 μm, about 0.5 μm to 1.2 μm, or about 0.6 μm to 1 μm, e.g., about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.3 μm, about 1.4 μm, about 1.5 μm, about 1.6 μm, about 1.7 μm, about 1.8 μm, about 1.9 μm, or about 2 μm. In specific embodiments, the focal-distance intervals are 0.5 μm. In some embodiments, methods of identifying a microbial species include acquiring a plurality of images at each focal length and combining the images.

In further embodiments, methods of identifying a microbial species include selecting an image having a greatest value of one or more quality metrics for measurement. The quality of the selected image can be improved by removing or smoothing numerical noise prior to segmentation. In some embodiments, methods of identifying a microbial species include

performing dimensionality reduction, e.g., PCA or UMAP, on the multidimensional distribution of measured parameters.

Using methods of identification as disclosed herein, measuring 100 randomly selected segmented microorganisms is sufficient to identify the microbial species with a confidence of about 93-97%.

In some embodiments, a sample of the microbial species originates from a growth detection study. The growth detection study may have been performed using the device as disclosed herein or a same or different sample plate.

In further embodiments, methods of identifying a microbial species include identifying a second microbial species.

In accordance with an aspect, methods of performing growth detection, identification and antimicrobial susceptibility testing (AST) on a microbial species are disclosed. The disclosed method is configured to perform all three functions in less than about eight hours.

In some embodiments, one or both of the growth detection of the microbial species and identification of the microbial species is performed using the methods disclosed herein.

In some embodiments, AST is based on a differential growth detection method, e.g., as disclosed herein. For example, the AST involves dilution temporal modeling (DTM). Using methods disclosed herein, AST achieves categorical agreement to a reference method in under one hour, e.g., under 1 hour, under 55 minutes, under 50 minutes, under 45 minutes, under 40 minutes, under 35 minutes, under 30 minutes, under 25 minutes, under 20 minutes, under 15 minutes, under 10 minutes, or under 5 minutes. In some embodiments, the reference method for AST comparison includes broth microdilution.

In some embodiments, the AST is performed with respect to an antibiotic selected from cefepime, meropenem, ciprofloxacin, and gentamicin. Any antibiotic can be used for AST as disclosed herein, and the choice of antibiotic is a function of the bacterial identification performed prior to AST. The methods of this disclosure are in no way limited by the choice of antibiotic used for AST.

In some embodiments, results pertaining to AST are reported to an operator, a laboratory information system, and/or an electronic medical record, e.g., transmitted by the device as disclosed herein or viewed using a user interface on the device as disclosed herein.

In some embodiments, AST may be performed once a microorganism has been identified at a categorical level but before the microorganism has been identified at the species level. In some embodiments, each of growth detection, identification, and AST are performed in a single device, e.g., a device as disclosed herein.

In accordance with an aspect, a quality control method for a pharmaceutical manufacturing process is disclosed. The quality control method includes performing the method of detecting growth of a microbial species, e.g., using any device or method disclosed herein, on a sample containing a pharmaceutical component or finished end product to assess a bioburden thereof.

In some embodiments, the quality control method for a pharmaceutical manufacturing process includes accepting or rejecting the pharmaceutical component or finished end product based on comparison of the assessed bioburden to a threshold value.

### EXAMPLES

The function and advantages of these and other embodiments can be better understood from the following examples. These examples are intended to be illustrative in nature and are not considered to be limiting the scope of the invention.

### Example 1

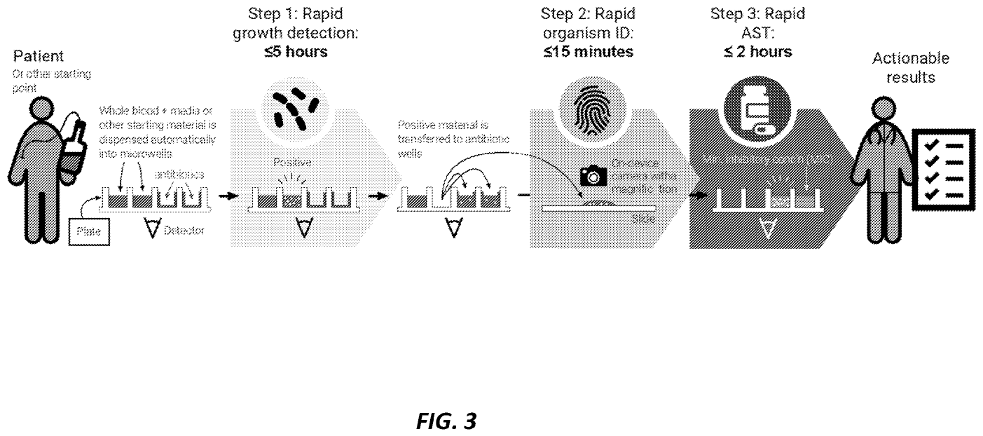

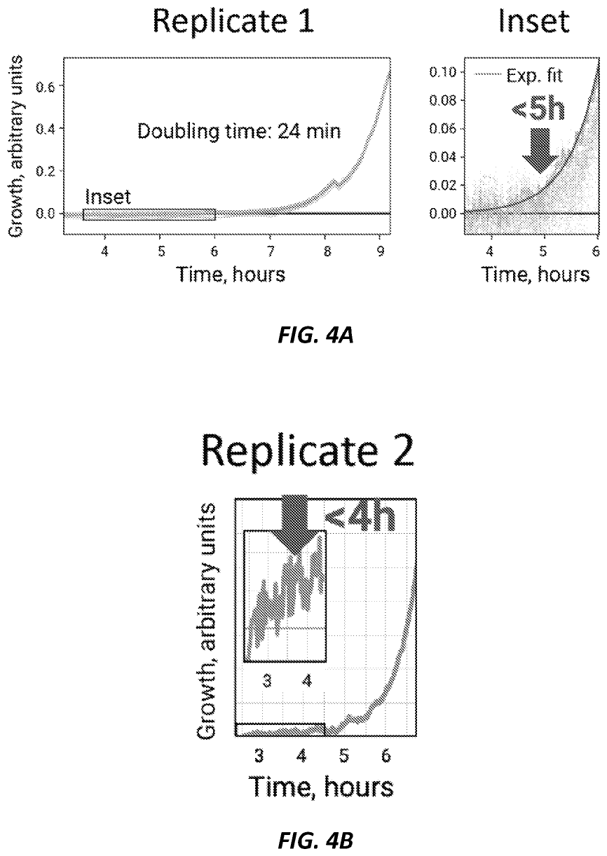

This example demonstrates the growth detection component of an embodiment of the disclosed system. Specifically, it demonstrates growth detection directly from whole blood in under five hours. FIG. 3 shows a typical experimental workflow. In the workflow shown, human blood was spiked with E. coli at quantities typical of septic patients (e.g., 1-10 CFU/mL), mixed with growth media, and automatically dispensed into the wells of a microwell plate. The number of wells was large, typically 384 or 1,536 wells, such that each well contained no more than a single bacterium purely by Poisson filling. This indicated that growth detection was digital. As a result, the number of wells with growth was proportional to the number of replication-competent bacterial (or other) cells (i.e., CFUs). Thus, this system allowed for the quantitation of bioburden. Each well was monitored for growth by automated RGB-sensitive imaging sensors. Sensor data from each well produced a time series. Each time series was processed and a statistically significant change from baseline was interpreted as growth. Processing included, but was not limited to, accounting for background changes in blood over time and exclusion of extreme values. FIGS. 4A-4B shows results from two experiments, demonstrating growth in under five hours and under four hours, respectively. In an embodiment, data from multiple wells can be compared to increase sensitivity. Differences in time series between wells (e.g., color or doubling time) are used to indicate suspicion of polymicrobial infection, which can be further investigated by identification.

### Example 2

This example demonstrates bacterial identification directly from an unstained smear. “Unstained,” as used herein, refer to material that was imaged without the addition of any stains and without undergoing any staining or other reagent-mediated enhancement procedure, such as gram staining. Bacteria were grown as described in EXAMPLE 1. A smear was made of material recovered from a well following growth detection as described in EXAMPLE 1. Making a smear constituted placing the liquid material on a glass slide, allowing the material to spread across the slide, and then heat-drying the material to fix the material to the slide.

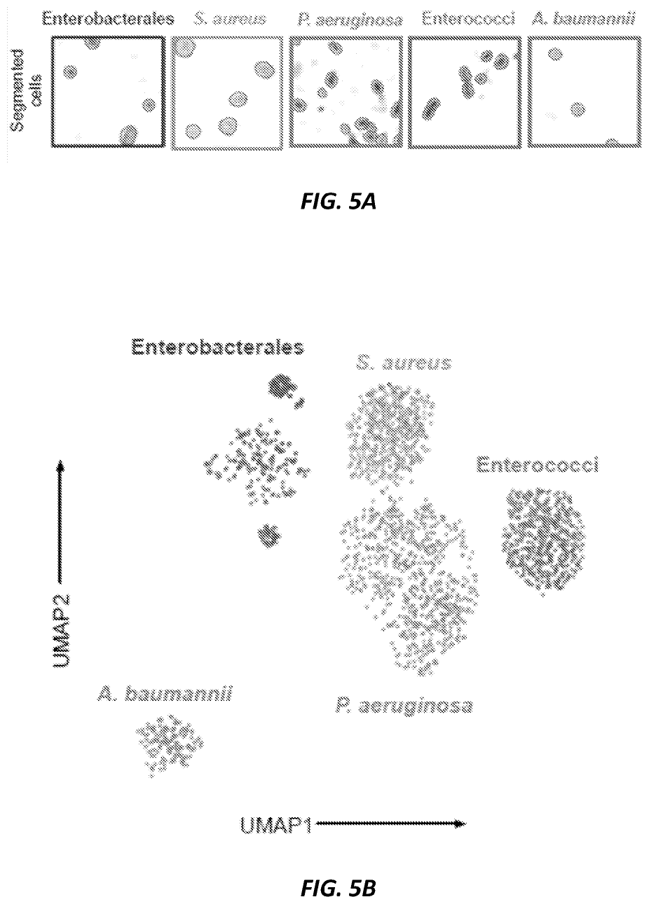

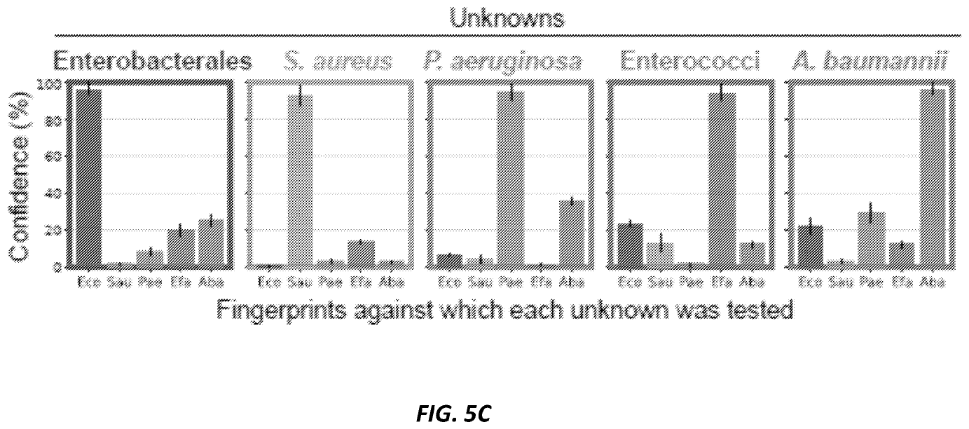

The slide was then imaged at 40× magnification. A blue filter was used to sharpen the images. In practice, the filter varied the color profile in a predictable manner while preserving useful color information. Images were taken at multiple focal lengths and at small/fine-grained focal-distance intervals of 0.5 μm. This distance was chosen as it was smaller than the diameter of a bacterial cell, ensuring at least one focal length will lead to in-focus images. Multiple images were taken automatically at each focal length. These images were then combined to substantially eliminate imaging noise. Multiple images were also taken without the slide, and additional multiple images were taken with the slide but without illumination. These were used to control for potential background artifacts to produce “clean” combined images from each focal length. Comparison of the clean combined images from different focal lengths allowed the most in-focus clean combined image to be selected. Bacterial cells and potential debris, such as remnants of lysed red blood cells), collectively referred to here as “objects,” were clearly visible on these images, despite the lack of stain as illustrated in FIGS. 5A-5C. At this point, further cleanup was performed using super-resolution techniques to sharpen images, for example using deep learning.

Each object in the microscopic field of view was segmented. FIG. 5A illustrates segmentation on cells from different bacterial species and groups. There were typically hundreds of objects per field, a plurality of which are bacterial cells. The cells were known to belong to the same bacterial species because the well from which the smear was made began with a single bacterial cell. Thus, there was no possibility that the cells segmented from a given smear came from multiple different microbial strains or species.

Next, a number of specific measurements were made of each segmented object. In this example, these measurements included the size of the object calculated by counting the number of pixels in the object and converting to standard units of area, e.g. μm², by knowing the dimensions of a pixel, the perimeter of each object similarly calculated in units of length, e.g. μm, the mean color of the object calculated both as red-green-blue values and as hue-lightness-saturation values, with each value between 0 and 255, and the number of neighbors each object has corrected for the total number of objects in the field, since a denser field will result in more neighbors just by chance. The number of neighbors and related measures were a quantitative way of assessing bacterial cell arrangement, which was useful for identification. For example, Staphylococci cluster and therefore each Staphylococcus cell had many neighbors whereas Enterococci do not cluster, meaning each Enterococcus cell had fewer neighbors. Thus, through measurement, each object became associated with an ordered series of numbers. In this example, these associations corresponded to size, perimeter, color, and relative arrangement. Mathematically, the term of art for this ordered series of numbers is a “vector:” the measurement step described in this paragraph was said to assign a vector to, or define a vector for, each object; the vector describes the object. Each object received its own vector. The set of vectors for all the objects segmented from the smear thereby was a quantitative description of the smear.

In this example, smears were made, segmented, and measured as described herein for each of five species or groups of bacteria: Staphylococcus aureus, Pseudomonas aeruginosa, Acinetobacter baumannii, Enterobacterales (the group that contains E. coli), and Enterococci (the group that contains Enterococcus faecium and Enterococcus faecalis) as illustrated in FIGS. 5A-5C. The set of vectors for each species or group defined that species or group. Each vector can be considered a point in space. If there are three measurements, this will be a point in 3-dimensional space, i.e., x,y,z of the Cartesian plane. The first measurement provided the distance in the x direction, the second provided the distance in the y direction, and the third provided the distance in the z direction. The set of vectors thereby corresponded to a “point cloud” in this space. Each point in the point cloud corresponded to measurements from a single bacterial cell. When there were more than 3 measurements, as in this example, the result was a point cloud in higher-dimensional space. To aid visualization of high-dimensional spaces, mathematical tools known as “dimensionality-reduction” techniques (e.g., Principal Component Analysis (PCA) and Uniform Manifold Approximation and Projection (UMAP)) were used to permit the higher dimensions to be “flattened” into 2 dimensions while preserving the overall shapes and relative arrangements of the point clouds. FIGS. 5A-5C further illustrate the results of dimensionality reduction using the UMAP technique on the above five species and groups of organisms. This figure demonstrates that corresponding point clouds had characteristic configurations. These configurations were “fingerprints” of each species. For example, the S. aureus fingerprint was self-contained, meaning S. aureus cells were relatively homogeneous in terms of their measurements. In contrast, the Enterococci fingerprint had two parts: a larger part which was self-contained and a smaller part which overlapped the S. aureus point cloud. This configuration indicated that while the vast majority of Enterococcus cells looked different from most S. aureus cells, a minority looked similar to S. aureus. To tell that a smear contained an Enterococcus and not S. aureus, enough cells had to be measured such that that some appeared in the larger part of the S. aureus point cloud. FIGS. 5A-5C shows that 100 cells were enough to identify unknowns with 93-97% confidence. In each panel of this figure, the vectors for 100 cells were selected randomly from a smear and compared to the fingerprints in FIG. 5B, and the probability (×100) that they corresponded to each fingerprint was shown as the height of the bars. In each case, the correct identification was made, shown as the tallest bar, and no incorrect identification was close, shown as the next-tallest bar. Thus, this example demonstrated high-confidence identification from unstained cells in the context of the integrated three-part (growth detection/ID/AST) invention described in this disclosure.

### Example 3

This example demonstrated categorical agreement in AST between an embodiment of the disclosed system and a reference method in under 1 hour. “Categorical agreement” is a term of art in AST that describes whether an organism is susceptible or resistant by each of two methods, typically a reference method and an investigative method. The reference method is broth microdilution and comes from the CLSI (M100).

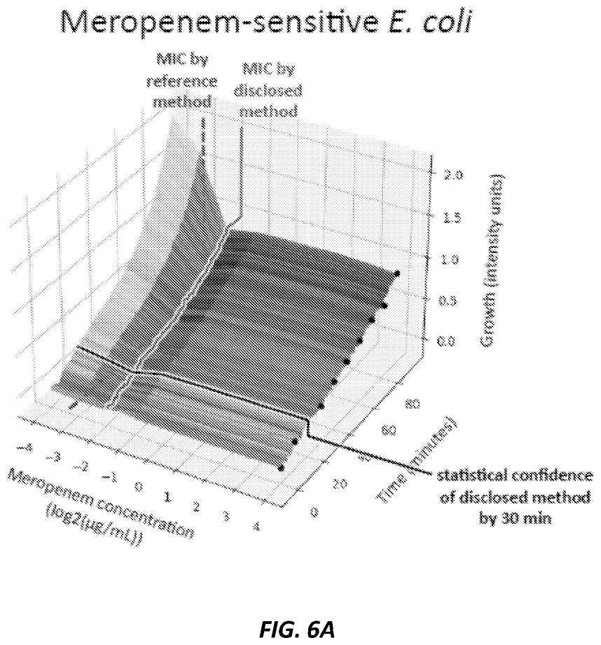

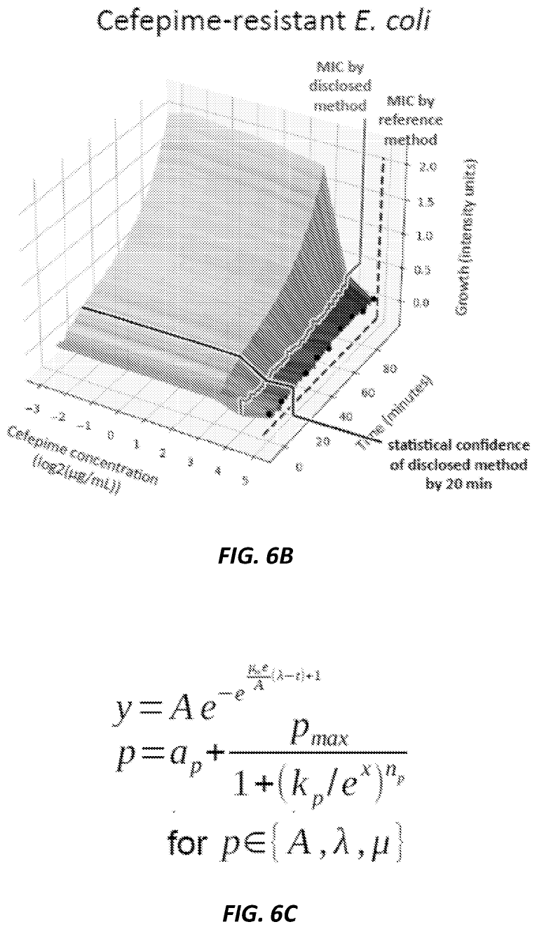

FIGS. 6A-6B shows results from a typical experiment. Each experiment involved a standard inoculum of bacteria grown at a concentration similar to what was recovered from the growth-detection step, which began with whole blood. The inoculum was automatically dispensed into the wells of a microwell plate. The wells contained doubling dilutions of standard antibiotics such as cefepime, meropenem, ciprofloxacin, and gentamicin (for gram-negative bacteria). A large number of wells enabled multiple replicates per condition, providing robustness to random dropouts or to growth variation.

In each experiment, the growth and color of each well was monitored for growth by an automated imager. Serial images for each “bug-drug” combination were combined to create a unified picture of bacterial growth for each antibiotic, measure MICs, and determine any correction factors necessary to maximize S/I/R categorical agreement with reference MICs. “Unified picture” refers to dilution-temporal modeling or DTM, in which the Gompertz model of growth over time with the Hill model of growth as a function of antibiotic concentration were combined to generate a comprehensive 3D picture of growth as a function of both time and concentration (FIG. 6A-6B). The equation for this modeling is shown in FIG. 6C. In the meropenem-susceptible strain of E. coli shown in FIG. 6A, a fall in growth with increased antibiotic concentration was clearly visible at 0.5 μg/mL. The resulting MIC by the disclosed method (red line), was in categorical agreement with, and within the single doubling dilution of, the reference-method MIC (blue line) that is allowed in clinical practice. This fall is discerned with statistical confidence at 30 minutes in this example (black line). In contrast, in the cefepime-resistant strain of E. coli shown in FIG. 6B, growth remained effectively unchanged as antibiotic concentration rises, until the concentration reached 16 μg/mL, whereupon there is a falloff, indicating the MIC by the disclosed method disclosed herein. Again, this is in categorical agreement with, and within a single doubling dilution of, the reference-method MIC. In this example, the fall is discerned with statistical confidence at 20 minutes.

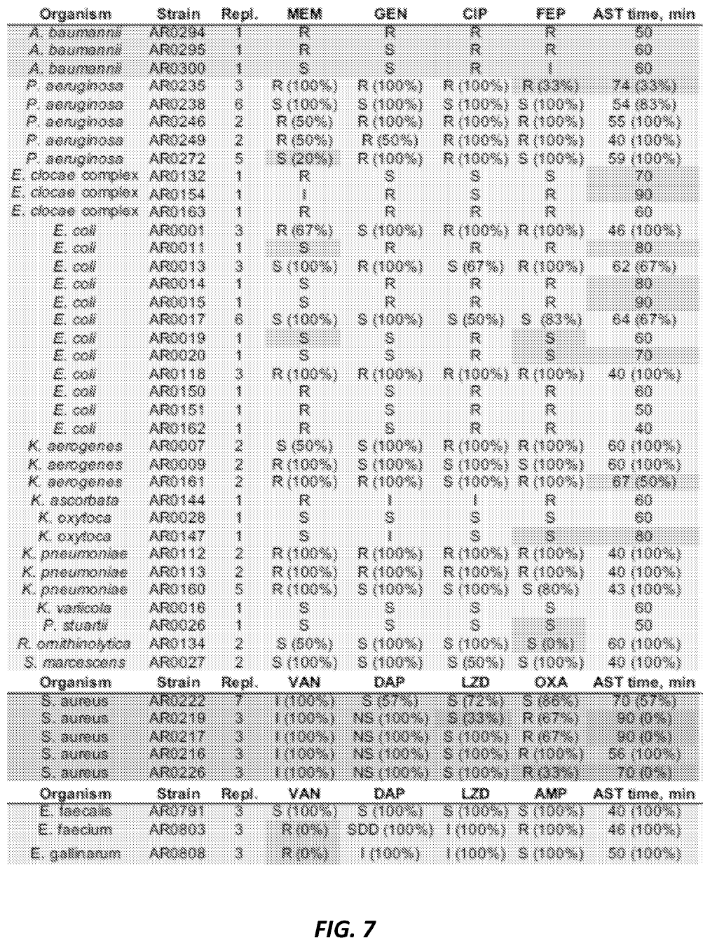

FIG. 7 shows a tally of results for 11 standard antibiotics, for organisms representing five key groups of bacteria: Enterococci, Staphylococci, Enterobacterales, Acinetobacter baumannii, and Pseudomonas aeruginosa. These groups include the especially important ESKAPE organisms and account for two-thirds to three-quarters of all bacterial bloodstream infections. Doubling times across multiple strains of Enterococci (including E. faecium and E. faecalis strains), Staphylococcus (including S. aureus), A. baumannii, P. aeruginosa, and Enterobacterales strains (including E. coli and Klebsiella) were typically 20-40 minutes, and usually 20-30 minutes. This is consistent with completing growth, ID, and AST in under 8 hours.

### Example 4

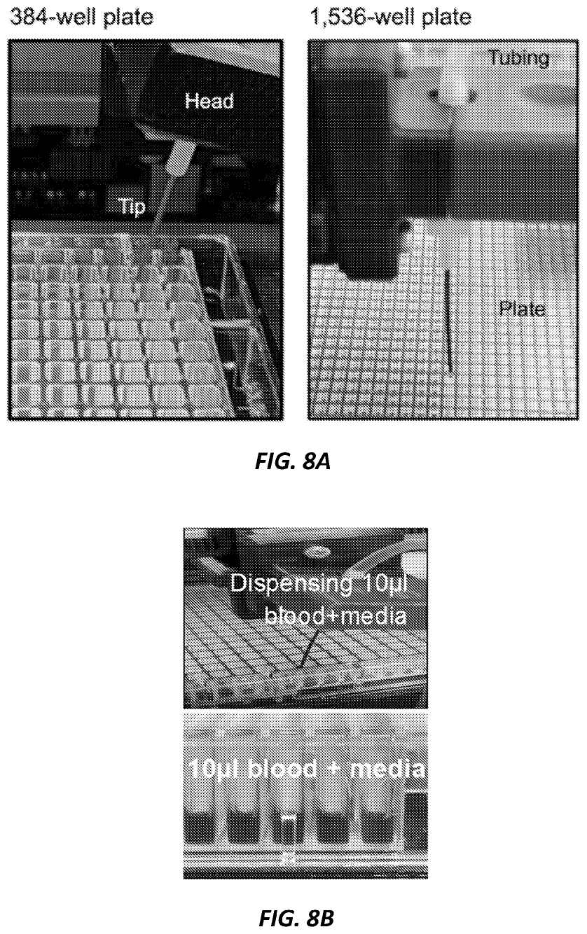

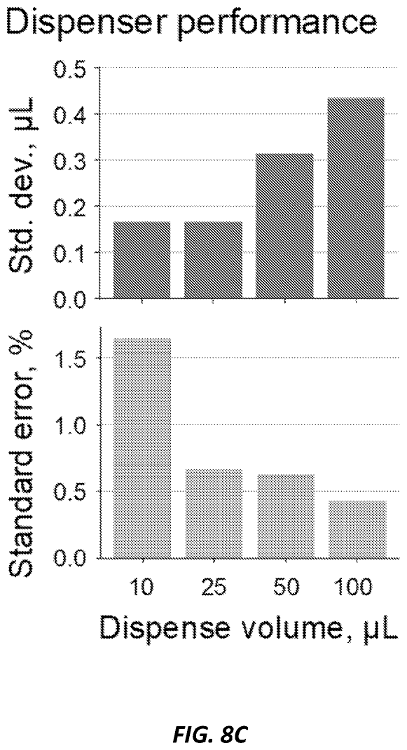

This example demonstrates automated fluid handling using a dispenser, which was used in the examples disclosed herein for several purposes. These purposes included filling microwells with a mixture of whole blood (or other starting specimen) and growth media (EXAMPLE 1), obtaining material from one or more microwells for ID (EXAMPLE 2), and filling microwells for AST (EXAMPLE 3). The dispenser was fast, filling about 1 well/second) and accurate. The dispenser included a flexible tip held in place by a head. The tip was connected to tubing through which the relevant fluid flowed as illustrated in FIGS. 8A-8B. Flow was controlled by a peristaltic pump. The pump used a two-lobe design with ball bearings to minimize friction with the tubing. Driving the pump was a stepper motor shaft with an absolute-position magnetic encoder. This magnetic encoder provided the lobes' angular position at any time, which made it possible to compensate for any position-related heterogeneity in flow and therefore provide very low standard deviation in the dispensed volume from well to well. A motorized gantry system moved the head and needle from well to well. FIGS. 8A-8B demonstrates the dispenser's use in filling either 384-well or 1,536-well microwell plates. The inset demonstrates dispensing 10 μL of blood-plus-growth media. It shows both that the dispenser could handle this material without clogging and that the result is precise filling. The bar plots illustrated in FIG. 8C demonstrate the dispenser's precision. Specifically, these bar plots showed a standard deviation of less than 0.2 μL, corresponding to a standard error of 1.6%, on dispense volumes as low as 10 μL, demonstrating excellent performance.

### Example 5

It was suggested that computer vision could be used to rapidly identify bacteria well enough to interpret AST results by training a deep neural network on a combination of Gram-stain images and broth-culture color. Gram staining stains bacteria pink (Gram-negative) or purple (Gram-positive), making their shape, size, and relative configuration visible under a microscope. Color is an attribute of macroscopic colonies on agar plates; it has been demonstrated that color can was detectable from broth culture using the sub-hour device disclosed herein. Gram stain and color are helpful for identification.

Deep learning is the mainstay of computer vision and has shown great promise for bacterial ID. Deep-learning-based classifiers learn by studying many images to find patterns unique to each of a set of classes, which for bacteria can be species (e.g., Enterococcus faecium), genera (Enterococci), or some other type of category (Gram-positive cocci). Existing datasets include 660 Gram-stain images from pure colonies of 24 Gram-positive and 9 Gram-negative bacteria was released that included E. faecium, E. faecalis, S. aureus, A. baumannii, P. aeruginosa, and E. coli. Existing deep-learning-based classifiers have reached >99% accuracy on this dataset. Deep learning has performed well on Gram stains from challenging real-world blood cultures, i.e., samples that have background detritus, variability in color and contrast, and crystallization artifacts, recently having achieved 94.9% accuracy classifying Gram-positive cocci in clusters (typical of Staphylococci), Gram-positive cocci in pairs and chains (Enterococci), and Gram-negative rods (A. baumannii, P. aeruginosa, and the Enterobacterales).

It was believed that a hierarchical approach, first classifying bacteria as Gram-positive or -negative and then performing ID within those groups, would translate best to the real world. Deep networks are powerful but can be fooled, and their decisions can be hard to understand. To make them more dependable and interpretable, the task of indemnification was split into stepwise tasks: classifying first by Gram-stain appearance and then by identification. Each task had its own classifier and confidence score, making decisions more accurate and interpretable.

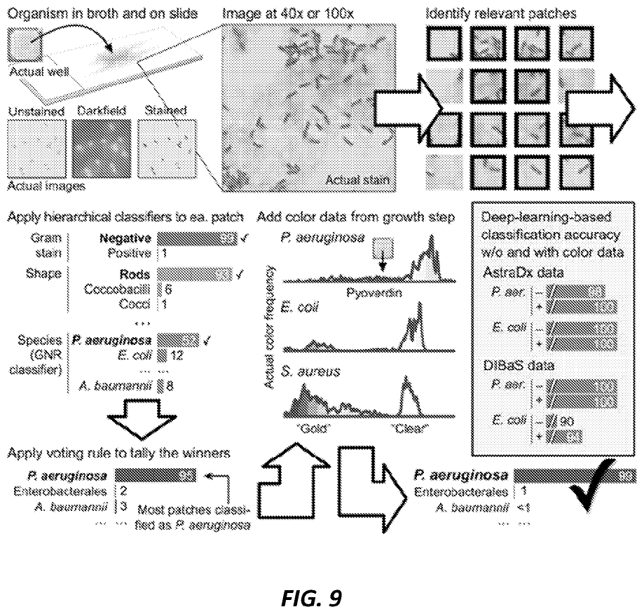

It was further believed that identifying bacteria singly or in small groups and then tallying the results would improve accuracy and interpretability. Classifiers often benefit from majority rules when the final decision depends on sub-decisions. This is often the case in medical imaging. For identification, each Gram-stain image was divided into small (80×80-pixel) patches that contain just about 1-20 bacteria cells each (allowing more than one so as to capture differences such as pairs-and-chains vs. clusters, palisading, etc.). Each patch was classified, and a vote winner was assigned as the final identification as illustrated in FIG. 9.

Gram-stain heterogeneity was informative in the clinical laboratory; for example, Gram-variable rods suggest Clostridia. The experiment disclosed in this example ensured that heterogeneity would always be a property of a single organism and not due to the presence of mixed organisms because each well started from a single cell. If majority rule proved insufficient, the information in the tallies was used to create more sophisticated rules, for example involving decision trees and/or the relative entropy between the observed tally and the reference distribution of a known bacterium.

It was additionally believed that using the color of unstained bacteria would aid in identification, especially in cases where different-colored organisms have similar Gram-stain appearances, such as shown in the box inset in FIG. 9. For example, P. aeruginosa and E. coli were essentially indistinguishable by eye on Gram stain. However, the green, blue, or purple colonies of P. aeruginosa for the clear-to-buff-colored colonies of E. coli were distinguished. It has been shown that the sub-hour device as disclosed herein detected not only subtle changes in growth but also subtle changes in color that are consistent with the known colors of organisms as illustrated in the center pane of FIG. 9. These included clear-to-yellow for E. coli and S. aureus and purple-to-green for P. aeruginosa that was due to pyocyanin and/or pyoverdin production. While not every strain was colored, color provided discriminatory information for identification for those that were colored.

In this example, a deep learning network was built and trained. The deep learning network used hierarchical classification, color, and a majority rule to demonstrate 100% Gram-stain accuracy and 95% identification accuracy including a set of Gram stain images connected in house as illustrated in FIG. 9. A fixed-objective 40×-magnification microscope was fabricated using a lens that could be precisely positioned in front of the sub-hour device's gantry-controlled camera. The camera could be moved from looking at the well plate to looking at a Gram-stain slide.

Each image was sliced into small patches that contained 1-10 bacteria cells each. The deep learning network disclosed herein showed color improved performance, as accuracy on E. coli and P. aeruginosa rose from 89% and 99% to x % and y %, respectively. Each classification took less than a second. The voting tallies appeared to carry information about whether an ID was likely to be correct, suggesting that more sophisticated rules would further improve performance. These results showed the strength of the stepwise approach disclosed herein and supported the use of machine vision and deep learning networks for bacterial identification as part of single-shift S2A.

### Prophetic Example 1

In a prophetic example of one embodiment, the design of a device that performs growth detection, ID, and AST on whole blood, for purposes of direct AST from whole blood in the setting of sepsis, as shown in FIGS. 2A-2C. Briefly, whole blood is to be collected into a receptacle that contains proprietary or other bacterial growth media. This receptacle is to be scanned by a barcode scanner to collect and confirm the patient and sample information. The sample is to be inverted and inserted into the device, which is a temperature-controlled and optionally air-tight enclosure. A disposable microwell plate that contains a region for performing smears is to be inserted via a door in the front of the device. The hands-on time for an operator to perform these steps is about 2 minutes. Upon insertion of the receptacle and plate, a needle or other retrieval device will enter the receptacle and the peristaltic pump draws material via tubing. This material is to be dispensed via a peristaltic pump-powered dispenser, which is to be operated by a moving gantry controlled by a microcontroller, into the microwells of the plate. Several wells will be left empty as controls. This process takes several minutes.

Step 1: growth detection: The plate is to be positioned by a plate holder between an RGB illuminator and a sensitive kHz-MHz detector array that will contain detectors that monitor each well. The plate is then monitored for growth. The illuminator cycles between red, green, and blue light at a frequency of approximately single-digit Hz, allowing the detector to perform many readings per cycle. These readings will constitute per-well times series. A program run by the microprocessor of the device will integrate the measurements into clean color information. In general, a decrease in light and/or change in color will be interpreted as growth. Comparison of the time series among the wells, between the wells and controls, and between wells and reference time series, will allow any background time-varying patterns, i.e., changes that are not associated with growth, to be subtracted off. Optionally, clustering of time series across wells may reveal two patterns, which may be interpretable as growth and no growth, respectively. Such cross-well comparisons may also be used to increase the sensitivity of growth detection beyond what is possible from single wells. In this context, an increase in sensitivity means detecting growth earlier by considering multiple wells than is possible by considering each well separately. In this example, growth detection will be expected to take less than or about five hours for the vast majority of clinically significant bacteria. The device's touchscreen will allow an operator to monitor the process, if or as desired.

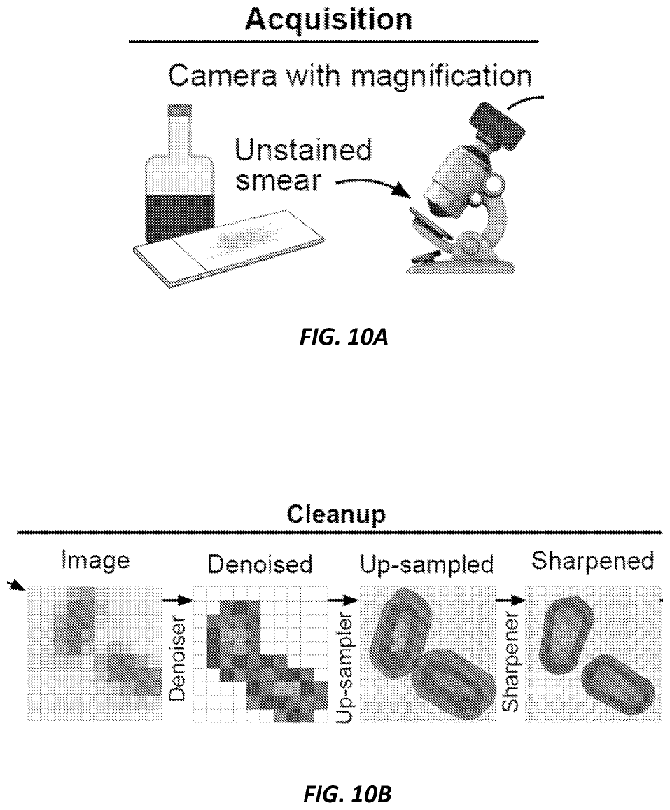

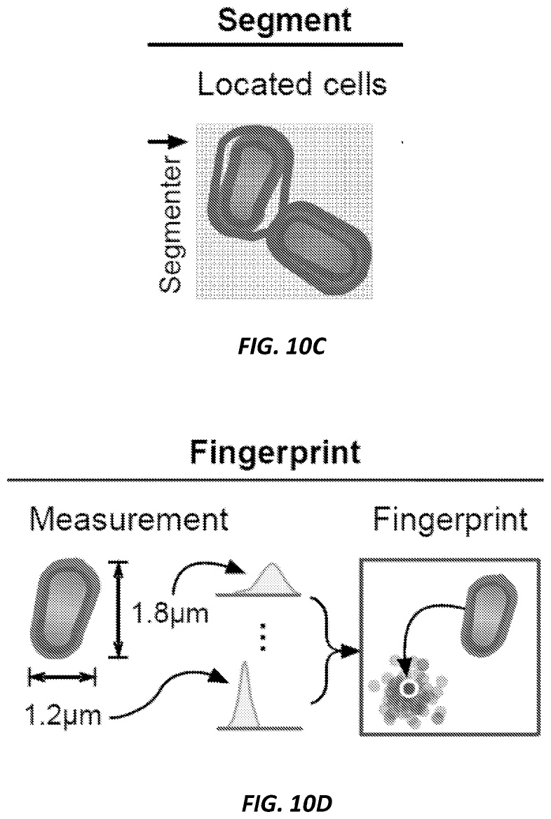

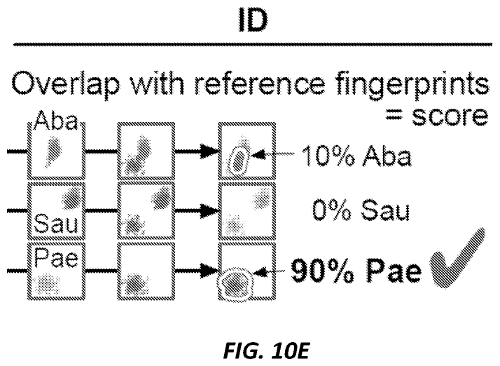

Step 2: Identification: Once growth is detected, the dispenser will serve as an aspirator, by reversing the direction of the pump, and positive material will be collected from one or more wells and dispensed into the smear area and the smear is dried by the internal heating components of the enclosure. A magnifying camera also on the moving head will then be positioned above the smear and the smear will be repeatedly imaged, directed by a program on the microprocessor. The head will be moved up and down through a range that includes the focal plane as illustrated in FIG. 10A. The microprocessor will then process the images, for example as described in EXAMPLE 2 and with optional further cleanup steps, e.g., via deep-learning-enabled super-resolution as illustrated in FIG. 10B. Objects will then be segmented as illustrated in FIG. 10C, measured, and fingerprinted as illustrated in FIG. 10D, and the fingerprint will be compared to reference fingerprints to render an identification as illustrated in FIG. 10E. Note that the comparison illustrated in FIG. 10E is to include comparison to fingerprints of blood debris (red-cell ghosts, platelets, white-cell nuclei, etc.) to avoid erroneous identification. Identification is expected to take ≤15 minutes. The information will then be reported to the operator via the user interface of the device, shown as a touchscreen. Optionally, if the time series show evidence of a second organism in a different set of wells, a second identification can be performed. The operator will be informed of the presence of a second organism. A sample of each may be returned to the operator at any time through the door of the device.

Step 3: AST: Finally, using the dispenser as disclosed herein, material from positive wells will be aspirated, optionally mixed with fresh media, and dispensed into antibiotic-containing wells on a different part of the plate. The plate will contain enough wells for replicates of 20 or more different antibiotics and/or antibiotic combination, including combinations with e.g., beta-lactamase inhibitors, at multiple doubling-dilution concentrations. As used herein, a “doubling dilution” is a series of concentrations such as 4 μg/mL, 2 μg/mL, and 1 μg/mL that are related by each subsequent dilution containing half the concentration of antibiotic as the previous dilution. The plate will be repositioned between the illuminator and detector of the device and imaged as described above, In this manner, what is being observed is differential growth detection, i.e., differences in growth in the presence of different concentrations of different antibiotics. An integrated picture of growth will be obtained as described in EXAMPLE 3, a MIC will be determined for each antibiotic or antibiotic combination, and results will be reported to the operator via the device's user interface. If in a hospital setting, results are also reported via the communication hardware to a laboratory information system (LIS), and via that system to the hospital's electronic medical record (EMR), providing actionable results to the provider.

### Prophetic Example 2

### Overview

In this prophetic example, the projected development and testing of a device that performs growth detection, bacterial identification, and AST in a single hospital shift is described. Specific aims are to develop each of these three components to a stated target performance threshold (Aims 1-3) and then combine them and test the resulting device (Aim 4).

### AIM 1. Demonstrate Rapid Growth Detection on the Photodiode-Based Device

Device. The initial prototype device disclosed herein is to be upgraded by moving the remaining components inside, simplifying the circuitry to reduce noise, and replacing the white LED with red, green, and blue LEDs to measure color and improve sensitivity. Preliminary studies demonstrated the ability to rapidly build and upgrade prototypes and to handle rapid on-and-off LED changes. These preliminary studies also demonstrated the utility of color for bacterial identification. The growth-detection experiments for Aim 1 will proceed as in Example 1, with blood, but with only 192 wells being filled (100 μL/well) and with rapid cycling through the red, green, and blue LEDs to allow the photodiode to record color changes. In addition to being useful for ID, color is likely to improve sensitivity, since color changes can precede intensity changes by as much as a doubling time, which was observed in growth experiments using the sub-hour device disclosed herein.

Microwell plate format. It is envisioned that the final device is to include a single 384- or 1,536-microwell plate, with half the wells dedicated for growth detection and the other half dedicated for AST. Therefore, our growth experiments and AST experiments each use only half a plate.

Experiments. Growth-detection experiments are to be run in triplicate on 100 extensively characterized strains from BEI/ATCC and the FDA-CDC Antimicrobial Resistance Bank (AR), the same strains we have been using. These will include 20 strains each from the five groups: Enterococci, Staphylococci, Enterobacterales, A. baumannii, and P. aeruginosa, including methicillin-resistant S. aureus (MRSA; both linezolid-sensitive and -resistant strains), vancomycin-resistant Enterococci (VRE), extended-spectrum beta-lactamase (ESBL)-producing E. coli, carbapenem-resistant Enterobacterales (CRE), carbapenem-resistant A. baumannii (CRAB), and carbapenem-resistant P. aeruginosa (CRPA). Enterobacterales will include Enterobacter, Serratia, Citrobacter, Morganella, and Proteus, all common causes of severe BSIs.

Performance threshold. The minimum performance threshold is to be able to detect 14 doublings starting from a single bacterium. This is a change of from 214 to about 16,000 bacteria/well, translating to 4 h40 for strains with a 20-minute doubling time. The sensitivity of the photodiode makes improving growth detection a matter of noise reduction. Each step taken has shown to reduce noise 2-5×. It is anticipated that an additional 2-3× improvement from the layout and circuitry upgrades and another 2× improvement by adding color, accounting for 2-doubling improvement, ˜2× better than necessary to reach the above-listed threshold.

Statistical rigor. Each experiment will be performed in triplicate.

Pitfalls and alternatives. Some bacteria divide slowly, and some blood contains growth inhibitors. Just as there is a long tail beyond the median for growth detection in current practice (e.g. Cutibacterium), a long tail beyond our anticipated 4-6-hour median is anticipated. However, additional options are available to assure that most bacterial strains are detected close to the median. These include combining data across wells to improve sensitivity, modifying the broth to enhance recovery, adding detection amplifiers such as intravital dyes, and using deeper 1,536-well plates to focus the signal under the general relationship that a similar volume and a quarter of the area should result in a ˜4× increase in the sensitivity. Together, these options are anticipated to provide for greater than or equal to 8× sensitivity, cutting an additional hour from growth detection for a 20-minute doubler.

### AIM 2. Demonstrate Rapid AST on the Photodiode Device for the Same Strains as Aim 1.

Rationale. The photodiode device's sensitivity should enable rapid AST even from the few bacteria available after rapid growth detection. Sub-hour AST starting from ˜10⁶ bacteria/well was demonstrated as disclosed herein. The sub-hour device as disclosed herein could not detect changes of less than 5×10⁵-1×10⁶ bacteria/well at sub-hour timescales because of low frame rate. It is expected that growth detection is to to culminate with ˜50 wells of 16,000 bacteria/well. Setting aside one well for identification (Aim 3) and dividing the rest among 192 AST wells will provide ˜4,000 bacteria cells for each AST well. The photodiode will need 2.3 doublings to detect growth starting from 4,000 cells, and other doubling to detect MICs for a total of 3.3 doublings, or slightly over an hour for a 20-minute doubling time, for a total S2A of 5-6 hours. It is anticipated that an S2A of ≤8 hours will be applicable for most bacterial strains.

Performance threshold. The threshold is AST within three hours, starting from this low inoculum.

Primary and secondary measures. Percent categorical (S/I/R) agreement is important for treating patients and is therefore the primary measure. It will be calculated in standard FDA fashion: the number of agreements divided by the total tested. Secondary measures are to include very major, major, and minor error rates, essential agreement (that is, agreement±1 doubling dilution), time to MIC, and k_{μm}, MIC, and confidences (with mean±s.d. for the triplicates).

Ground truth. The gold-standard reference MIC comparator will be determined by CLSI reference broth microdilution (CLSI M07) and interpreted categorically according to CLSI guidelines (CLSI M100).

Testing and validation. To determine the rules for calling MICs, 40 strains are to be tested in triplicate from each of the five groups (200 total instead of the 100 in Aim 1 to test a range of susceptibility patterns). These rules will then be validated on an additional 200 strains.

Experiments. Experiments will be as described in the Examples herein but with four modifications. First, the experiments will start with a lower inoculum of about 4,000 bacteria per well. Second, starting material will be prepared in cation-adjusted Mueller Hinton broth spiked with 12% blood to simulate material recovered from positive wells after growth detection. Third, only half the wells on the plate will be used. And fourth, there will be 18 antibiotics instead of 11, with two replicates for each of five doubling-dilution concentrations, improvements resulting from the photodiode device's improved sensitivity. The concentrations will bracket the most common MIC across the most common bacteria. Twelve wells will be left as positive and negative controls (no antibiotic and broth only/empty wells, respectively).

Antibiotics. The antibiotics will be the following CLSI front-line agents: ampicillin, clindamycin, daptomycin, doxycycline, linezolid, oxacillin, and vancomycin (for Gram positives); ampicillin/sulbactam, cefepime, ceftazidime, ceftazidime-avibactam, ceftriaxone, gentamicin, and meropenem (Gram negatives); and cefazolin, cefoxitin, levofloxacin, and trimethoprim/sulfamethoxazole (both). These will be pre-loaded and freeze-dried onto the plates.

Analysis. The DTM model as disclosed herein will be used to calculate and measure k_{μm} at the earliest timepoint. The MIC for each drug will be measured as the concentration immediately above the k_{μm}. For example, if k_{μm}=0.79 μg/mL (between the 0.5 and 1 μg/mL dilutions), the MIC will be 1 μg/mL. If the k_{μm} is outside the tested range, the MIC will be recorded as > or <the relevant limit (e.g., >64 μg/mL). Note that on modern platforms, raw MICs sometimes require internal adjustment to agree with reference MICs, for example subtracting one dilution for a particular antibiotic. As necessary, drug-, group-, and species-specific rules will be developed to correct the MICs, to maximize categorical agreement.

Statistical rigor. 200 strains in triplicate are consistent with the FDA's 510(k) guidelines for statistical confidence. Bootstrapping will be used to provide confidence intervals on k_{μm}. Detection time will be defined as the earliest timepoint at which we can detect growth in the positive-control wells and see, for strains that exhibit dose dependence, a monotonic dose-dependent trend that is robust to bootstrapping or, for strains with high-level resistance, growth equivalent to the positive control across doses.

Pitfalls and alternatives. Although the current design will detect mixed cultures, a second plate would be necessary to perform AST on them. In this situation, the original plate is to be used for AST on the additional organism(s) in the remaining wells.

AIM 3. Demonstrate ID from Photomicrographs and Bacterial Color.

Approach. The plan is to continue to develop the hierarchical and majority-rule approach. Briefly, 40×- or 100×-magnification photomicrographs will be split into small patches that contain bacteria. These will be classified as Gram positive vs. gram negative, cocci vs. rods vs. coccobacilli, etc. Within these categories, they will be further classified using the appropriate genus or species-level categories, such as Enterobacterales vs. P. aeruginosa vs. A. baumannii for Gram-negative rods. Results across all patches will be integrated via majority vote or similar rule to assign the final ID. Note that although identification to the species level has been demonstrated, AST interpretation requires identification only to the level of a CLSI category, often broader than a specific species (e.g., Enterobacterales).

Model architectures. The following model architectures will be tested: CNNs EfficientNet, ConvNex, and RepLKNet and the transformer Swin as part of neural-based decision trees (NBDTs) These CNNs and the transformer are the among the current best performers on image-classification tasks. NBDTs learn an entire hierarchy of labels at once (Gram positive, coccus, clusters, yellow) instead of just a final label (S. aureus). They enable end-to-end learning while retaining hierarchy/multiple labels and human interpretability. An NBDT will provide results that a particular patch was successful, for example, bacteria>detritus: 99%, Gram positive>Gram negative: 99%, cocci>rod: 99%, clusters>pairs-and-chains: 94%, yellow>white 99%, and therefore S. aureus: 95%; many 95% S. aureus patches→>99% S. aureus overall. The addition of layers to integrate voting will also be explored.

Training and validation sets. In deep learning, large, heterogeneous training sets are the key to generalizability. 200 strains will be imaged in triplicate experiments as outlined in Aims 1 and 2. The images will be collected at image fields of 40× and 100×. Each imaged field is expected to contain tens to hundreds of bacteria. For further heterogeneity, Gram stains will be prepared in replicate by hand and by our device's system (Aim 4) and will be imaged by a microscope as well as the camera having a photodiode as disclosed herein. This dataset will thus have heterogeneity of species, strains, cameras, magnification, staining, and fields. Each Gram stain will be reviewed by a human for quality control and then labeled according to the various characteristics of the strain. Standardized color will be added as HSV or RGB triples from the growth-detection and AST experiments. The dataset will be split by strain into training and validation sets, so that no strain appears in both sets. In addition, images from the public dataset will be added in to the model validation set to further assess generalizability. In both training and validation, patches will be generated automatically.

On-device Gram stain. An on-device Gram staining module will be constructed that will utilizer the fluid handling system disclosed herein, the heating element disclosed herein (for drying the slide), the existing camera having the photodiode detector (with auto focus), and the existing magnification lens as disclosed herein. Training will be performed using a cloud-based service, e.g., Amazon Web Services, but classification (inference) is not computationally intensive and so will be performed on the device's internal computer.

Statistical rigor. This experiment is expected to produce a large and diverse training set and a separate validation set. To test for robustness, each fit will be repeated 10-20 times and the stability of results between fits compared using the Jaccard index. Model performance will be measured by accuracy (correctly classified samples÷total samples), both overall and for each category.

Performance threshold. One goal of this experiment is 99% accuracy to the level of CLSI categories required for interpreting AST. Note that even state-of-the-art ID systems have trouble with certain pairs, for example E. coli vs. Salmonella strains on MALDI.

### AIM 4. Build the Integrated Photodiode Device, Ready for Premarket Field Evaluation.

Components. The photodiode-based growth detector and fluid handler as disclosed herein with be integrated together with a Gram-stain component for bacterial identification into a single device. Preliminary studies have de-risked these components or demonstrated their operability outright. A receptacle for the blood-culture bottle, a mechanism for removing/replacing plate lids, and a touchscreen interface with the necessary GUI and software will also be constructed and added.

### Expected Outcomes

This proposal will result in a device that will lower the median S2A from two days to 6-8 hours. The expected outcome is therefore a meaningful step toward reducing mortality from bloodstream infections for people around the world.

The phraseology and terminology used herein is for the purpose of description and should not be regarded as limiting. As used herein, the term “plurality” refers to two or more items or components. The terms “comprising,” “including,” “carrying.” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. A device configured to perform growth detection, identification, and antimicrobial susceptibility testing (AST) on a microbial species, the device comprising:
a housing configured to receive a sample plate;
a sample port configured to receive a sample suspected to contain the microbial species;
a fluid distribution system constructed and arranged to introduce the sample to one or more sample wells of the sample plate;
a sample plate imaging system; and
a controller configured to collect data from the sample plate imaging system and further configured to process the collected data to:
detect growth of the microbial species;
identify the microbial species; and
perform AST on the microbial species.

2. The device of claim 1, wherein the fluid distribution system comprises a gantry, a fluid dispensing head operatively coupled to the gantry, and a pump fluidly connected to the fluid dispending head and comprising a stepper motor shaft with an absolute-position magnetic encoder.

3. The device of any of the preceding claims, wherein the fluid distribution system is constructed and arranged to facilitate digital growth detection.

4. The device of any of the preceding claims, wherein the sample plate imaging system comprises:
a camera configured with optics, the camera connected to the fluid dispensing head;
a light source; and
detector array.

5. The device of any of the preceding claims, wherein the detector array comprises a photodetector, e.g., a charge coupled device (CCD), a complementary metal oxide semiconductor (CMOS) detector, or a photodiode.

6. The device of any of the preceding claims, wherein the device further comprises a user interface.

7. The device of any of the preceding claims, wherein the controller is configured to transmit information pertaining to growth, identification, and/or AST to a user.

8. The device of any of the preceding claims, wherein the device further comprises a heater to facilitate preparation of a sample.

9. The device of any of the preceding claims, wherein the device is further constructed and arranged to enable Gram staining on the sample.

10. The device of any of the preceding claims, wherein the device is constructed and arranged to perform all three functions in less than about eight hours.

11. The device of any of the preceding claims, wherein the device is constructed and arranged to perform all three functions in less than about six hours.

12. The device of any of the preceding claims, wherein the device performs AST in less than about one hour.

13. The device of any of the preceding claims, wherein the device a same approach for both growth detection and AST.

14. The device of any of the preceding claims, wherein identification of the microbial species is based on a stainless approach.

15. The device of any of the preceding claims, wherein the microbial species comprises a bacterial species.

16. The device of any of the preceding claims, wherein the microbial species is selected from the genus Acinetobacter, Escherichia, Klebsiella, Pseudomonas, Enterococcus, Streptococcus, and Staphylococcus.

17. The device of any of the preceding claims, wherein the microbial species falls within one of the following groups of bacteria: Enterococci, Staphylococci, Enterobacterales, Acinetobacter baumannii, and Pseudomonas aeruginosa.

18. The device of any of the preceding claims, wherein the microbial species comprises a nonbacterial species of fungus, mycobacteria, stool parasite, blood parasite or tissue parasite.

19. The device of any of the preceding claims, wherein the sample is a whole blood sample of a subject.

20. The device of any of the preceding claims, wherein the sample is associated with a pharmaceutical manufacturing component or finished end product.

21. The device of any of the preceding claims, wherein the device is configured to perform one or more of growth detection, identification and AST on a second microbial species.

22. A kit comprising the device of any of the preceding claims and a sample plate.

23. The kit of claim 22, wherein the sample plate includes a first portion for growth detection and a second portion for AST.

24. The kit of claim 23, wherein one or more wells in the second portion of the sample plate are preloaded with a freeze-dried antibiotic.

25. The kit of claim 24, wherein the antibiotic is preloaded according to a serial dilution scheme for AST.

26. The kit of any one of claims 22-25, wherein the sample plate contains a region for performing sample smears.

27. The kit of any one of claims 22-26, wherein one or more wells of the sample plate are defined by a geometry selected to facilitate sensitivity.

28. The kit of any one of claims 22-27, wherein the sample plate contains 384 or 1536 wells.

29. The kit of any one of claims 22-28, further comprising a source of a growth media or a detection amplifier.

30. The kit of any one of claims 22-29, further comprising a source of a Gram stain.

31. The kit of any one of claims 22-29, further comprising a sample bottle.

32. A method of detecting growth of a microbial species, comprising:
providing a sample suspected to contain the microbial species;
acquiring a time series of images of the sample; and
detecting growth of the microbial species via time dependent change pertaining to at least one of light and color across the time series of images.

33. The method of claim 32, wherein detecting growth comprises detecting growth using RGB-sensitive imaging sensors in a device.

34. The method of claim 32 or 33, wherein a statistically significant change from a baseline pertaining to at least one of light and color is interpreted as growth of the microbial species.

35. The method of any preceding claim, wherein a decrease in light and/or a change in color across the time series of images is interpreted as growth of the microbial species.

36. The method of any preceding claim, further comprising acquiring a time series of images for each well of a sample plate and detecting growth in each well.

37. The method of any preceding claim, wherein detecting growth further comprises comparing one or more of: the time series of images among the wells, between the wells and controls, or between wells a reference time series.

38. The method of any preceding claim, further comprising comparing data across multiple wells to increase sensitivity of growth detection.

39. The method of any preceding claim, wherein growth detection is achieved in a duration of under five hours.

40. The method of any preceding claim, wherein a number of wells of a sample plate with observable growth is used to determine a number of replication-competent microbial cells.

41. The method of any preceding claim, further comprising quantifying a bioburden of the sample based on growth detection.

42. The method of claim 40, further comprising taking remedial action based on quantitation of bioburden.

43. The method of any preceding claim, further comprising subjecting the microbial species to identification and/or AST upon detecting growth.

44. The method of any preceding claim, further comprising detecting polymicrobial infection by comparing the time series of images between wells of a sample plate.

45. The method of claim 43, wherein the presence of polymicrobial infection is evaluated based on a difference in color or doubling time across wells of the microwell plate.

46. The method of any preceding claim, further comprising processing the time series of images to increase a quality thereof to facilitate earliest possible detection of growth.

47. The method of any preceding claim, further comprising adding growth media or a detection amplifier to the sample.

48. The method of any preceding claim, wherein the sample is a whole blood sample.

49. The method of any preceding claim, wherein the sample is associated with a pharmaceutical manufacturing component or finished end product.

50. The method of any preceding claim, wherein the method is characterized by digital growth detection.

51. A method of identifying a microbial species, comprising:
imaging a sample comprising a plurality of microorganisms of the microbial species to obtain a series of polymicrobial images;
segmenting the polymicrobial images;
measuring parameters of each segmented microorganism to provide a multidimensional distribution of measured parameters; and
classifying the microbial species based on the multidimensional distribution of measured parameters.

52. The method of claim 51, wherein the measured parameters pertain to size, shape, intrinsic color, arrangement and other morphological properties of the microbial species.

53. The method of claim 51 or 52, wherein at least one of the measured parameters is selected from the group consisting of: width, length, interior density, membrane thickness, color heterogeneity, color concentration, curvature, tapering, aspect ratio, and concavity.

54. The method of any of claims 51-53, wherein the method comprises collecting inherent color data associated with the microbial species without staining.

55. The method of any of claims 51-54, wherein classification involves machine learning trained with at least one of the following modalities: (i) unstained slides imaged with direct light; (ii) unstained slides imaged with indirect light; and (iii) pairing pre- and post-stained images.

56. The method of any of claims 51-55, wherein classifying the microbial species involves a probability distribution.

57. The method of any of claims 51-56, wherein classifying is performed via a hierarchical approach.

58. The method of any of claims 51-57, comprising distinguishing gram positive bacteria from gram negative bacteria with a first confidence score.

59. The method of claim 58, wherein the species of each bacteria is then identified with a second confidence score.

60. The method of any of claims 51-59, wherein classification is performed via majority rule, decision tree or relative entropy between an observed tally and a reference distribution of a known microbial species.

61. The method of any of claims 51-60, further comprising filtering the images.

62. The method of claim 61, wherein a blue filter is applied to the images.

63. The method of any of claims 51-62, wherein images of each sample are taken at multiple focal lengths.

64. The method of any of claims 51-63, wherein images of each sample are taken at a series of small or fine-grained focal-distance intervals.

65. The method of claim 64, wherein the focal-distance intervals are 0.5 μm.

66. The method of any of claims 51-65, further comprising acquiring a plurality of images at each focal length and combining the images.

67. The method of any of claims 51-66, further comprising selecting an image having a greatest value of one or more quality metrics for measurement.

68. The method of any of claims 51-67, further comprising increasing a quality of the selected image by removing or smoothing numerical noise prior to segmentation.

69. The method of any of claims 51-68, further comprising performing dimensionality reduction on the multidimensional distribution of measured parameters.

70. The method of any of claims 51-69, wherein measuring 100 randomly selected segmented microorganisms is sufficient to identify the microbial species with a confidence of about 93-97%.

71. The method of any of claims 51-70, wherein a sample of the microbial species originates from a growth detection study.

72. The method of any of claims 51-71, further comprising identifying a second microbial species.

73. A method of performing growth detection, identification and antimicrobial susceptibility testing (AST) on a microbial species, wherein the method is configured to perform all three functions in less than about eight hours.

74. The method of claim 73, wherein growth detection of the microbial species is performed according to any of the preceding claims.

75. The method of claim 73 or 74, wherein identification of the microbial species is performed according to any of the preceding claims.

76. The method of any of claims 73-75, wherein AST is based on a differential growth detection method.

77. The method of claim 76, wherein AST involves dilution temporal modeling (DTM).

78. The method of claim 76 or 77, wherein AST achieves categorical agreement to a reference method in under one hour.

79. The method of any of claims 76-78, wherein the reference method involves broth microdilution.
A single well: number of wells/total volume of sample that went on the plate (eg in mL)=number of replication-competent cells (eg CFU, for “colony-forming unit”) per unit volume (eg CFU/mL) about a

80. The method of any of claims 76-79, wherein AST is with respect to an antibiotic selected from the group consisting of: cefepime, meropenem, ciprofloxacin, and gentamicin.

81. The method of any of claims 76-80, wherein results pertaining to AST are reported to an operator, a laboratory information system, and/or an electronic medical record.

82. The method of any of claims 76-81, wherein AST may be performed once a microorganism has been identified at a categorical level but before the microorganism has been identified at the species level.

83. The method of any of claims 73-82, wherein the three functions are performed in a single device.

84. A quality control method for a pharmaceutical manufacturing process, comprising:
performing the method of detecting growth of a microbial species of any of the preceding claims on a sample containing a pharmaceutical component or finished end product to assess a bioburden thereof.

85. The method of claim 84, further comprising accepting or rejecting the pharmaceutical component or finished end product based on comparison of the assessed bioburden to a threshold value.
